(19) United States
(12) Patent Application Publication
(10) Pub. No.: US 2020/0211713 A1
(43) Pub. Date: Jul. 2, 2020

(54) METHOD AND SYSTEM TO CHARACTERIZE DISEASE USING PARAMETRIC FEATURES OF A VOLUMETRIC OBJECT AND MACHINE LEARNING

(71) Applicant: Analytics For Life Inc., Toronto (CA)

(72) Inventors: Ian Shadforth, Morrisville, NC (US); Timothy William Fawcett Burton, Toronto (CA); Sunny Gupta, Belleville (CA); Farhad Fathieh, North York (CA)

(21) Appl. No.: 16/725,402

(22) Filed: Dec. 23, 2019

Related U.S. Application Data
(60) Provisional application No. 62/835,869, filed on Apr. 18, 2019.
(60) Provisional application No. 62/784,984, filed on Dec. 26, 2018.

(51) Int. Cl. G16H 50/50; G16H 10/60; G16H 50/20; G16H 15/00; G06N 20/00; G06N 7/00; G06T 15/08; G06T 7/00; G06T 7/13; A61B 5/00; A61B 5/02

(52) U.S. Cl. CPC G16H 50/50; G16H 10/60; G16H 50/20; G16H 15/00; G06N 20/00; G06N 7/005; G06T 2207/30076; G06T 7/0012; G06T 7/13; A61B 5/7267; A61B 5/02007; G06T 2207/10072; G06T 15/08

(57) ABSTRACT

The exemplified methods and systems employs non-invasively acquired biophysical measurements of a subject in a residue analysis that is structured as a three-dimensional volumetric object to which machine extractable features associated with a geometric associated aspect of the three-dimensional volumetric object may be derived and used for in the training and/or prediction of a disease state. The system generates a residue model from a point-cloud residue generated from an analysis of the plurality of biophysical signal data sets. The system generates a three-dimensional volumetric object from the point-cloud residue from which machine extractable features associated with the point-cloud residue maybe extracted.

### RELATED APPLICATION

This U.S. patent application claims priority to, and the benefit of, U.S. Patent Provisional Application No. 62/784,984, filed Dec. 26, 2018, entitled “Method and System to Assess Disease Using Phase Space Tomography and Machine Learning,” and U.S. Patent Provisional Application No. 62/835,869, filed Apr. 18, 2019, entitled “Method and System to Assess Disease Using Phase Space Tomography and Machine Learning,” each of which is incorporated by reference herein in its entirety.

### FIELD OF THE INVENTION

The present disclosure generally relates to non-invasive methods and systems for characterizing cardiovascular circulation and other physiological system. More specifically, in an aspect, the present disclosure relates to non-invasive methods that utilize acquired biophysical signal (e.g., a cardiac signal, a brain/neurological signal, signals associated with other biological systems, etc.) and using that biophysical signal in the prediction and localization of cardiac and/or non-cardiac disease and pathologies.

### BACKGROUND

Ischemic heart disease, also known as cardiac ischemia or myocardial ischemia, is a disease or group of diseases characterized by a reduced blood supply to the heart muscle, usually due to coronary artery disease (CAD). CAD typically occurs when the lining inside the coronary arteries that supply blood to the myocardium, or heart muscle, develops atherosclerosis (the hardening or stiffening of the lining and the accumulation of plaque therein, often accompanied by abnormal inflammation). Over time, CAD can also weaken the heart muscle and contribute to, e.g., angina, myocardial infarction (cardiac arrest), heart failure, and arrhythmia. An arrhythmia is an abnormal heart rhythm and can include any change from the normal sequence of electrical conduction of the heart and in some cases can lead to cardiac arrest.

The evaluation of CAD can be complex, and many techniques and tools are used to assess the presence and severity of the condition. In the case of electrocardiography, a field of cardiology in which the heart's electrical activity is analyzed to obtain information about its structure and function, significant ischemic heart disease can alter ventricular conduction properties of the myocardium in the perfusion bed downstream of a coronary artery narrowing or occlusion. This pathology can express itself at different locations of the heart and at different stages of severity, making an accurate diagnosis challenging. Further, the electrical conduction characteristics of the myocardium may vary from person to person, and other factors such as measurement variability associated with the placement of measurement probes and parasitic losses associated with such probes and their related components can also affect the biophysical signals that are captured during electrophysiologic tests of the heart. Further still, when conduction properties of the myocardium are captured as relatively long cardiac phase gradient signals, they may exhibit complex nonlinear variability that cannot be efficiently captured by traditional modeling techniques.

There is a benefit to having additional tools to non-invasively evaluate coronary artery disease and other cardiac disease, neurological disease, and other disease of other physiological systems.

### SUMMARY

The exemplified methods and systems employs non-invasively acquired biophysical measurements of a subject in a residue analysis that generates a point cloud data set that is then structured as a three-dimensional volumetric object to which machine extractable features associated with a geometric associated aspect of the three-dimensional volumetric object may be derived and used for in the training and/or classification of a disease state.

In some embodiments, the analysis is used to facilitate the isolation or estimation of behaviors of the subject's physiological system as a dynamical system for the evaluation and/or prediction of presence of a disease state.

The generation of a point-cloud residue data set from an underlying analysis and the generation of three-dimensional volumetric object to derived machine extractable parameterized features lend itself well to machine learning analysis. Indeed, clinically-pertinent information about the physiological system (e.g., the heart) may be framed as the residue model that can be expressed in two- or three-dimensional point cloud analysis to which machine extractable parameterized features can be readily extracted.

As used herein, the term “cardiac signal” refers to one or more signals associated with the structure, function and/or activity of the cardiovascular system—including aspects of that signal's electrical/electrochemical conduction—that, e.g., cause contraction of the myocardium. A cardiac signal may include, in some embodiments, electrocardiographic signals such as, e.g., those acquired via an electrocardiogram (ECG) or other modalities.

As used herein, the term “neurological signal” refers to one or more signals associated with the structure, function and/or activity of the central and peripheral nervous systems, including the brain, spinal cord, nerves, and their associated neurons and other structures, etc., and including aspects of that signal's electrical/electrochemical conduction. A neurological signal may include, in some embodiments, electroencephalographic signals such as, e.g., those acquired via an electroencephalogram (EEG) or other modalities.

A “biophysical signal” is not limited to a cardiac signal, a neurological signal, or a photoplethysmographic signal but encompasses any physiological signal from which information may be obtained. Not intending to be limited by example, one may classify biophysical signals into types or categories that can include, for example, electrical (e.g., certain cardiac and neurological system-related signals that can be observed, identified and/or quantified by techniques such as the measurement of voltage/potential, impedance, resistivity, conductivity, current, etc. in various domains such as time and/or frequency), magnetic, electromagnetic, optical (e.g. signals that can be observed, identified and/or quantified by techniques such as reflectance, interferometry, spectroscopy, absorbance, transmissivity, visual observation, photoplethysmography, and the like), acoustic, chemical, mechanical (e.g., signals related to fluid flow, pressure, motion, vibration, displacement, strain), thermal, and electrochemical (e.g. signals that can be correlated to the presence of certain analytes, such as glucose). Biophysical signals may in some cases be described in the context of a physiological system (e.g., respiratory, circulatory (cardiovascular, pulmonary), nervous, lymphatic, endocrine, digestive, excretory, muscular, skeletal, renal/urinary/excretory, immune, integumentary/exocrine and reproductive systems), an organ system (e.g., signals that may be unique to the heart and lungs as they work together), or in the context of tissue (e.g., muscle, fat, nerves, connective tissue, bone), cells, organelles, molecules (e.g., water, proteins, fats, carbohydrates, gases, free radicals, inorganic ions, minerals, acids, and other compounds, elements and their subatomic components. Unless stated otherwise, the term “biophysical signal acquisition” generally refers to any passive or active means of acquiring a biophysical signal from a physiological system, such as a mammalian or non-mammalian organism. Passive and active biophysical signal acquisition generally refers to the observation of natural or induced electrical, magnetic, optical, and/or acoustics emittance of the body tissue. Non-limiting examples of passive and active biophysical signal acquisition means include, e.g., voltage/potential, current, magnetic, optical, acoustic and other non-active ways of observing the natural emittance of the body tissue, and in some instances, inducing such emittance. Non-limiting examples of passive and active biophysical signal acquisition means include, e.g., ultrasound, radio waves, microwaves, infrared and/or visible light (e.g., for use in pulse oximetry or photoplethysmography), visible light, ultraviolet light and other ways of actively interrogating the body tissue that does not involve ionizing energy or radiation (e.g., X-ray). Active biophysical signal acquisition may involve excitation-emission spectroscopy (including, e.g., excitation-emission fluorescence). Active biophysical signal acquisition may also involve transmitting ionizing energy or radiation (e.g., X-ray) (also referred to as “ionizing biophysical signal”) to the body tissue. Passive and active biophysical signal acquisition means can be performed with conjunction with invasive procedures (e.g., via surgery or invasive radiologic intervention protocols) or non-invasively (e.g., via imaging).

The methods and systems described in the various embodiments herein are not so limited and may be utilized in any context of another physiological system or systems, organs, tissue, cells, etc. of a living body. By way of example only, two biophysical signal types that may be useful in the cardiovascular context include cardiac signals that may be acquired via conventional electrocardiogram (ECG/EKG) equipment, bipolar wide-band biopotential (cardiac) signals that may be acquired from other equipment such as those described herein, and signals that may be acquired by various plethysmographic techniques, such as, e.g., photoplethysmography.

In the context of the present disclosure, techniques for acquiring and analyzing biophysical signals are described in particular for use in diagnosing the presence, non-presence, localization (where applicable), and/or severity of certain disease states or conditions in, associated with, or affecting, the cardiovascular (or cardiac) system, including for example pulmonary hypertension (PH), coronary artery disease (CAD), and heart failure (e.g., left-side or right-side heart failure).

Pulmonary hypertension, heart failure, and coronary artery disease are three diseases/conditions affiliated with the cardiovascular or cardiac system. Pulmonary hypertension (PH) generally refers to high blood pressure in the arteries of the lungs and can include a spectrum of conditions. PH typically has a complex and multifactorial etiology and an insidious clinical onset with varying severity. PH may progress to complications such as right heart failure and in many cases is fatal. The World Health Organization (WHO) has classified PH into five groups or types. The first PH group classified by the WHO is pulmonary arterial hypertension (PAH). PAH is a chronic and currently incurable disease that, among other things, causes the walls of the arteries of the lungs to tighten and stiffen. PAH requires at a minimum a heart catheterization for diagnosis. PAH is characterized by vasculopathy of the pulmonary arteries and defined, at cardiac catheterization, as a mean pulmonary artery pressure of 25 mm Hg or more. One form of pulmonary arterial hypertension is known as idiopathic pulmonary arterial hypertension—PAH that occurs without a clear cause. Among others, subcategories of PAH include heritable PAH, drug and toxin induced PAH, and PAH associated with other systemic diseases such as, e.g., connective tissue disease, HIV infection, portal hypertension, and congenital heart disease. PAH includes all causes that lead to the structural narrowing of the pulmonary vessels. With PAH, progressive narrowing of the pulmonary arterial bed results from an imbalance of vasoactive mediators, including prostacyclin, nitric oxide, and endothelin-1. This leads to an increased right ventricular afterload, right heart failure, and premature death. The second PH group as classified by the WHO is pulmonary hypertension due to left heart disease. This group of disorders is generally characterized by problems with the left side of the heart. Such problems can, over time, lead to changes within the pulmonary arteries. Specific subgroups include left ventricular systolic dysfunction, left ventricular diastolic dysfunction, valvular disease and, finally, congenital cardiomyopathies and obstructions not due to valvular disease. Treatments of this second PH group tends to focus on the underlying problems (e.g., surgery to replace a heart valve, various medications, etc.). The third PH group as classified by the WHO is large and diverse, generally relating to lung disease or hypoxia. Subgroups include chronic obstructive pulmonary disease, interstitial lung disease, sleep breathing disorders, alveolar hypoventilation disorders, chronic high-altitude exposure, and developmental lung disease. The fourth PH group is classified by the WHO as chronic thromboembolic pulmonary hypertension, caused when blood clots enter or form within the lungs, blocking the flow of blood through the pulmonary arteries. The fifth PH group is classified by the WHO as including rare disorders that lead to PH, such as hematologic disorders, systemic disorders such as sarcoidosis that have lung involvement, metabolic disorders, and a subgroup of other diseases. The mechanisms of PH in this fifth group are poorly understood.

PH in all of its forms can be difficult to diagnose in a routine medical examination because the most common symptoms of PH (shortness of breath, fatigue, chest pain, edema, heart palpitations, dizziness) are associated with so many other conditions. Blood tests, chest x-rays, electro- and echocardiograms, pulmonary function tests, exercise tolerance tests, and nuclear scans are all used variously to help a physician to diagnose PH in its specific form. As noted above, the “gold standard” for diagnosing PH, and for PAH in particular, is a cardiac catherization of the right side of the heart by directly measuring the pressure in the pulmonary arteries. If PAH is suspected in a subject, one of several investigations may be performed to confirm the condition, such as electrocardiography, chest radiography, and pulmonary function tests, among others. Evidence of right heart strain on electrocardiography and prominent pulmonary arteries or cardiomegaly on chest radiography is typically seen. However, a normal electrocardiograph and chest radiograph cannot necessarily exclude a diagnosis of PAH. Further tests may be needed to confirm the diagnosis and to establish cause and severity. For example, blood tests, exercise tests, and overnight oximetry tests may be performed. Yet further, imaging testing may also be performed. Imaging testing examples include isotope perfusion lung scanning, high resolution computed tomography, computed tomography pulmonary angiography, and magnetic resonance pulmonary angiography. If these (and possibly other) non-invasive investigations support a diagnosis of PAH, right heart catheterization typically is needed to confirm the diagnosis by directly measuring pulmonary pressure. It also allows measurement of cardiac output and estimation of left atrial pressure using pulmonary arterial wedge pressure. While non-invasive techniques exist to determine whether PAH may exist in a subject, these techniques cannot reliably confirm a diagnosis of PAH unless an invasive right heart catherization is performed. Aspects and embodiments of methods and systems for assessing PH are disclosed in commonly-owned U.S. patent application Ser. No. 16/429,593, the entirety of which is hereby incorporated by reference.

Heart failure affects almost 6 million people in the United States alone, and more than 870,000 people are diagnosed with heart failure each year. The term “heart failure” (sometimes referred to as congestive heart failure or CHF) generally refers to a chronic, progressive condition or process in which the heart muscle is unable to pump enough blood to meet the needs of the body, either because the heart muscle is weakened or stiff or because a defect is present that prevents proper circulation. This results in, e.g., blood and fluid backup into the lungs, edema, fatigue, dizziness, fainting, rapid and/or irregular heartbeat, dry cough, nausea and shortness of breath. Common causes of heart failure are coronary artery disease (CAD), high blood pressure, cardiomyopathy, arrhythmia, kidney disease, heart defects, obesity, tobacco use and diabetes. Diastolic heart failure (DHF), left- or left-sided heart failure/disease (also referred to as left ventricular heart failure), right- or right-sided heart failure/disease (also referred to as right ventricular heart failure) and systolic heart failure (SHF) are common types of heart failure.

Left-sided heart failure is further classified into two main types: systolic failure (or heart failure with reduced ejection fraction or reduced left ventricular function) and diastolic failure/dysfunction (or heart failure with preserved ejection fraction or preserved left ventricular function). Procedures and technologies commonly used to determine if a patient has left-sided heart failure include cardiac catheterization, x-ray, echocardiogram, electrocardiogram (EKG), electrophysiology study, radionucleotide imaging, and various treadmill tests, including a test that measures peak VO₂. Ejection fraction (EF), which is a measurement expressed as a percentage of how much blood a ventricle pumps out with each contraction (and in the case of left-sided heart failure the left ventricle), is most often obtained non-invasively via an echocardiogram. A normal left ventricular ejection fraction (LVEF) ranges from about 55% to about 70%.

When systolic failure occurs, the left ventricle cannot contract forcefully enough to keep blood circulating normally throughout the body, which deprives the body of a normal supply of blood. As the left ventricle pumps harder to compensate, it grows weaker and thinner. As a result, blood flows backwards into organs, causing fluid buildup in the lungs and/or swelling in other parts of the body. Echocardiograms, magnetic resonance imaging, and nuclear medicine scans (e.g., multiple gated acquisition) are techniques used to noninvasively measure ejection fraction (EF), expressed as a percentage of the volume of blood pumped by the left ventricle relative to its filling volume to aid in the diagnosis of systolic failure. In particular, left ventricular ejection fraction (LVEF) values below 55% indicate the pumping ability of the heart is below normal, and can in severe cases be measured at less than about 35%. In general, a diagnosis of systolic failure can be made or aided when these LVEF values are below normal.

When diastolic heart failure occurs, the left ventricle has grown stiff or thick, losing its ability to relax normally, which in turn means that the lower left chamber of the heart is unable to properly fill with blood. This reduces the amount of blood pumped out to the body. Over time, this causes blood to build up inside the left atrium, and then in the lungs, leading to fluid congestion and symptoms of heart failure. In this case, LVEF values tend to be preserved within the normal range. As such, other tests, such as an invasive catheterization may be used to measure the left ventricular end diastolic pressure (LVEDP) to aid in the diagnosis of diastolic heart failure as well as other forms of heart failure with preserved EF. Typically, LVEDP is measured either directly by the placement of a catheter in the left ventricle or indirectly by placing a catheter in the pulmonary artery to measure the pulmonary capillary wedge pressure. Such catheterization techniques, by their nature, increase the risk of infection and other complications to the patient and tend to be costly. As such, non-invasive methods and systems for determining or estimating LVEDP in diagnosing the presence or non-presence and/or severity of diastolic heart failure as well as myriad other forms of heart failure with preserved EF are desirable. In addition, non-invasive methods and systems for diagnosing the presence or non-presence and/or severity of diastolic heart failure as well as myriad other forms of heart failure with preserved EF, without necessarily including a determination or estimate of an abnormal LVEDP, are desirable. Embodiments of the present disclosure address all of these needs.

Right-sided heart failure often occurs due to left-sided heart failure, when the weakened and/or stiff left ventricle loses power to efficiently pump blood to the rest of the body. As a result, fluid is forced back through the lungs, weakening the heart's right side, causing right-sided heart failure. This backward flow backs up in the veins, causing fluid to swell in the legs, ankles, GI tract and liver. In other cases, certain lung diseases such as chronic obstructive pulmonary disease and pulmonary fibrosis can cause right-sided heart failure, despite the left side of the heart functioning normally. Procedures and technologies commonly used to determine if a patient has left-sided heart failure include a blood test, cardiac CT scan, cardiac catheterization, x-ray, coronary angiography, echocardiogram, electrocardiogram (EKG), myocardial biopsy, pulmonary function studies, and various forms of stress tests such as a treadmill test.

Pulmonary hypertension is closely associated with heart failure. As noted above, PAH (the first WHO PH group) can lead to an increased right ventricular afterload, right heart failure, and premature death. PH due to left heart failure (the second WHO PH group) is believed to be the most common cause of PH.

Ischemic heart disease, also known as cardiac ischemia or myocardial ischemia, and related condition or pathologies may also be estimated or diagnosed with the techniques disclosed herein. Ischemic heart disease is a disease or group of diseases characterized by a reduced blood supply to the heart muscle, usually due to coronary artery disease (CAD). CAD is closely related to heart failure and is its most common cause. CAD typically occurs when the lining inside the coronary arteries that supply blood to the myocardium, or heart muscle, develops atherosclerosis (the hardening or stiffening of the lining and the accumulation of plaque therein, often accompanied by abnormal inflammation). Over time, CAD can also weaken the heart muscle and contribute to, e.g., angina, myocardial infarction (cardiac arrest), heart failure, and arrhythmia. An arrhythmia is an abnormal heart rhythm and can include any change from the normal sequence of electrical conduction of the heart and in some cases can lead to cardiac arrest. The evaluation of PH, heart failure, CAD and other diseases and/or conditions can be complex, and many invasive techniques and tools are used to assess the presence and severity of the conditions as noted above. In addition, the commonalities among symptoms of these diseases and/or conditions as well as the fundamental connection between the respiratory and cardiovascular systems—due to the fact that they work together to oxygenate the cells and tissues of the body—point to a complex physiological interrelatedness that may be exploited to improve the detection and ultimate treatment of such diseases and/or conditions. Conventional methodologies to assess these biophysical signals in this context still pose significant challenges in giving healthcare providers tools for accurately detecting/diagnosing the presence or non-presence of such diseases and conditions.

For example, in electrocardiography—a field of cardiology in which the heart's electrical activity is analyzed to obtain information about its structure and function—it has been observed that significant ischemic heart disease can alter ventricular conduction properties of the myocardium in the perfusion bed downstream of a coronary artery narrowing or occlusion, the pathology can express itself at different locations of the heart and at different stages of severity, making an accurate diagnosis challenging. Further, the electrical conduction characteristics of the myocardium may vary from person to person, and other factors such as measurement variability associated with the placement of measurement probes and parasitic losses associated with such probes and their related components can also affect the biophysical signals that are captured during electrophysiologic tests of the heart. Further still, when conduction properties of the myocardium are captured as relatively long cardiac phase gradient signals, they may exhibit complex nonlinear variability that cannot be efficiently captured by traditional modeling techniques.

In an aspect, a method is disclosed for non-invasively assessing a representation of a physiological system in which the representation is indicative of a disease state of a subject, the method including obtaining, by one or more processors, a plurality of biophysical signal data sets of a subject; generating, by the one or more processors, a residue model comprising a point-cloud residue data set generated from an analysis of the plurality of biophysical signal data sets; generating, by the one or more processors, a three-dimensional volumetric object from the point-cloud residue data stet, wherein the point-cloud residue data set comprises a plurality of vertices defined in a three-dimensional phase space of the plurality of biophysical signal data sets; and determining, by the one or more processors, machine extractable features associated with a geometric associated aspect of the three-dimensional volumetric object, wherein the one or more machine extractable features are used as an indicator of a disease state.

In some embodiments, the step of generating the three-dimensional volumetric object comprises performing a triangulation operation on the point-cloud residue of the plurality of biophysical signal data sets, wherein the triangulation operation is selected from the group consisting of Delaunay triangulation, Mesh generation, Alpha Hull triangulation, and Convex Hull triangulation.

In some embodiments, the machine extractable features are used in a machine-trained estimation of presence and/or non-presence of significant coronary artery disease.

In some embodiments, the machine extractable features are selected from the group consisting of a 3D object volume value, a void volume value, a surface area value, a principal curvature direction value, and a Betti number value.

In some embodiments, the method further includes generating a contour data set for each tomographic data set/image of the set of tomographic data sets/images (e.g., wherein the contour data are presented for the assessment of presence and/or non-presence of significant coronary artery disease).

In some embodiments, the acquired plurality of biophysical signal data sets are derived from measurements acquired via the noninvasive equipment configured to measure properties selected from the group consisting of electric properties, magnetic properties, acoustic properties, impedance properties, and reflectance properties of a physiological system.

In some embodiments, the method further includes removing, by the one or more processors, a baseline wandering trend from the acquired data prior to generating the plurality of models.

In some embodiments, the method includes causing, by the one or more processors, generation of a visualization of generated volumetric object as a three-dimensional object, wherein the three-dimensional object is rendered and displayed at a display of a computing device and/or presented in a report.

In some embodiments, each of the acquired biophysical signal data sets comprises a wide-band phase gradient biopotential signal data set that is simultaneously acquired at a sampling rate selected from the group consisting of about 1 kHz, about 2 kHz, about 3 kHz, about 4 kHz, about 5 kHz, about 6 kHz, about 7 kHz, about 8 kHz, about 9 kHz, about 10 kHz, and greater than 10 kHz.

In some embodiments, the residue model is generated by a subtraction operation of the acquired biophysical signal data sets and a data set generated from the analysis of the plurality of biophysical signal data sets.

In some embodiments, the analysis of the plurality of biophysical signal data sets comprises a quasi-periodic analysis of the frequency components of the plurality of biophysical signal data sets.

In some embodiments, the analysis of the plurality of biophysical signal data sets comprises a chaotic analysis of the frequency components of the plurality of biophysical signal data sets.

In some embodiments, the analysis of the plurality of biophysical signal data sets comprises a phase analysis of the plurality of biophysical signal data sets.

In another aspect, a system is disclosed comprising a processor and a memory having instructions thereon, wherein the instructions when executed by the processor, cause the processor to perform any of the above method.

In another aspect, a non-transitory computer readable medium is disclosed having instructions stored thereon, wherein execution of the instructions, cause the processor to perform any of the above method.

### BRIEF DESCRIPTION OF THE DRAWINGS

The accompanying drawings, which are incorporated in and constitute a part of this specification, illustrate embodiments and together with the description, serve to explain the principles of the methods and systems. The patent or application file contains at least one drawing executed in color. Copies of this patent or patent application publication with color drawing(s) will be provided by the Office upon request and payment of the necessary fee.

Embodiments may be better understood from the following detailed description when read in conjunction with the accompanying drawings. The drawings include the following figures:

FIG. 1 is a diagram of an exemplary system configured to generate a residue model to non-invasively assess a physiological system to predict and/or estimate presence or non-presence of disease in such physiological system, in accordance with an illustrative embodiment.

FIG. 2A shows a plot of an example volumetric object generated from a residue point cloud model/data set representing an analysis of a cardiac system that has a positive diagnosis (i.e., unhealthy assessment) for presence of significant coronary artery disease, in accordance with an illustrative embodiment.

FIGS. 2B, 2C, 2D, 2E, 2F, 2G are example images as different projected views of the residue point cloud model/data set of FIG. 2A, in accordance with an illustrative embodiment.

FIG. 2H shows, in phase space, the pre-processed biophysical signal data set (e.g., corresponding to the input signal) used to generate the residue point cloud model/data set of FIG. 2A, in accordance with an illustrative embodiment.

FIG. 3A shows a plot of another example residue point cloud model/data set representing the behavior of another cardiac system that has a positive diagnosis (i.e., unhealthy assessment) for presence of significant coronary artery disease, in accordance with an illustrative embodiment.

FIGS. 3B, 3C, 3D, 3E, 3F, and 3G shows images generated from the residue point cloud model/data set of FIG. 3A, in accordance with an illustrative embodiment.

FIG. 3H shows, in phase space, the pre-processed biophysical signal data set (corresponding to the input signal) used to generate the residue point cloud model/data set of FIG. 3A, in accordance with an illustrative embodiment.

FIGS. 4A and 5A each shows a plot of example residue model/data set representing the behavior of cardiac systems that have a negative diagnosis (e.g., a healthy assessment) for presence of significant coronary artery disease, in accordance with an illustrative embodiment.

FIGS. 4B-4G and FIGS. 5B-5G show the corresponding images generated from the residue point cloud model/data sets of FIGS. 4A and 5A, respectively, in accordance with an illustrative embodiment.

FIGS. 4H and 5H show, in phase space, the pre-processed biophysical signal data set that is used to generate the residue point cloud model/data sets of FIGS. 4A and 5A, respectively, in accordance with an illustrative embodiment.

FIG. 6 is a diagram of an exemplary method of performing generating a volumetric object from a point-cloud residue of an analysis of a physiological system in accordance with an illustrative embodiment.

FIG. 7A is a diagram of a set of time-series plots of an example acquired cardiac signal data set acquired from the non-invasive measurement system of FIG. 1, in accordance with an illustrative embodiment.

FIG. 7B is a diagram of the example cardiac signal data set (e.g., wide-band phase gradient cardiac signal data set) of FIG. 7A shown in the frequency domain, in accordance with an illustrative embodiment.

FIGS. 8A and 8B are diagrams showing an example placement of surface electrodes as probes at the chest and back of a patient or subject to acquire biopotential signals associated with a cardiac signal data set, in accordance with an illustrative embodiment.

FIGS. 9A, 9B, and 9C are diagrams showing an example placement of surface electrodes as probes at the head and neck of a patient or subject to acquire biopotential signals associated with a neurological signal data set, in accordance with an illustrative embodiment.

FIG. 10 is a diagram showing an example of an acquired neurological signal data set and its corresponding representation in phase space, in accordance with an illustrative embodiment.

FIG. 11 shows a table of diagnostic performance of predictors using the exemplary system of FIG. 1, in accordance with an illustrative embodiment.

FIG. 12 shows a phase space plot that illustrates a set of subspace signals (e.g., associated with the pre-processed biophysical signal data set) and corresponding subspace model generated from a modeling module (from which the analysis module can generate the residue model/data set), in accordance with an illustrative embodiment.

FIG. 13 shows experimental results of differing residue pattern generated based on number of selected basis functions used in the subspace model, in accordance with an illustrative embodiment.

FIG. 14 shows experimental results of a residue model/data set generated by modeling frequencies in the input subspace signal using the Fast Fourier Transform, in accordance with an illustrative embodiment.

FIGS. 15A, 15B, 15C, 15D, 15E, and 15F shows show an example outputted classification for the presence and non-presence of significant coronary artery disease as determined via a neural network classifier, in accordance with an illustrative embodiment.

FIGS. 16A, 16B, 146C, 16D, 16E, and 16F show an example outputted classification overlaid with a contour data set and heat map associated with the classifier in accordance with an illustrative embodiment.

FIG. 17 shows an exemplary computing environment in which example embodiments of the assessment system and aspects thereof may be implemented.

### DETAILED SPECIFICATION

Each and every feature described herein, and each and every combination of two or more of such features, is included within the scope of the present invention provided that the features included in such a combination are not mutually inconsistent.

While the present disclosure is directed to the beneficial assessment of biophysical signals in the diagnosis and treatment of cardiac-related pathologies and conditions and/or neurological-related pathologies and conditions, such assessment can be applied to the diagnosis and treatment (including, surgical, minimally invasive, and/or pharmacologic treatment) of any pathologies or conditions in which a biophysical signal is involved in any relevant system of a living body. One example in the cardiac context is the diagnosis of CAD and its treatment by any number of therapies, alone or in combination, such as the placement of a stent in a coronary artery, performance of an atherectomy, angioplasty, prescription of drug therapy, and/or the prescription of exercise, nutritional and other lifestyle changes, etc. Other cardiac-related pathologies or conditions that may be diagnosed include, e.g., arrhythmia, congestive heart failure, valve failure, pulmonary hypertension (e.g., pulmonary arterial hypertension, pulmonary hypertension due to left heart disease, pulmonary hypertension due to lung disease, pulmonary hypertension due to chronic blood clots, and pulmonary hypertension due to other disease such as blood or other disorders), as well as other cardiac-related pathologies, conditions and/or diseases. Non-limiting examples of neurological-related diseases, pathologies or conditions that may be diagnosed include, e.g., epilepsy, schizophrenia, Parkinson's Disease, Alzheimer's Disease (and all other forms of dementia), autism spectrum (including Asperger syndrome), attention deficit hyperactivity disorder, Huntington's Disease, muscular dystrophy, depression, bipolar disorder, brain/spinal cord tumors (malignant and benign), movement disorders, cognitive impairment, speech impairment, various psychoses, brain/spinal cord/nerve injury, chronic traumatic encephalopathy, cluster headaches, migraine headaches, neuropathy (in its various forms, including peripheral neuropathy), phantom limb/pain, chronic fatigue syndrome, acute and/or chronic pain (including back pain, failed back surgery syndrome, etc.), dyskinesia, anxiety disorders, conditions caused by infections or foreign agents (e.g., Lyme disease, encephalitis, rabies), narcolepsy and other sleep disorders, post-traumatic stress disorder, neurological conditions/effects related to stroke, aneurysms, hemorrhagic injury, etc., tinnitus and other hearing-related diseases/conditions and vision-related diseases/conditions.

Example System

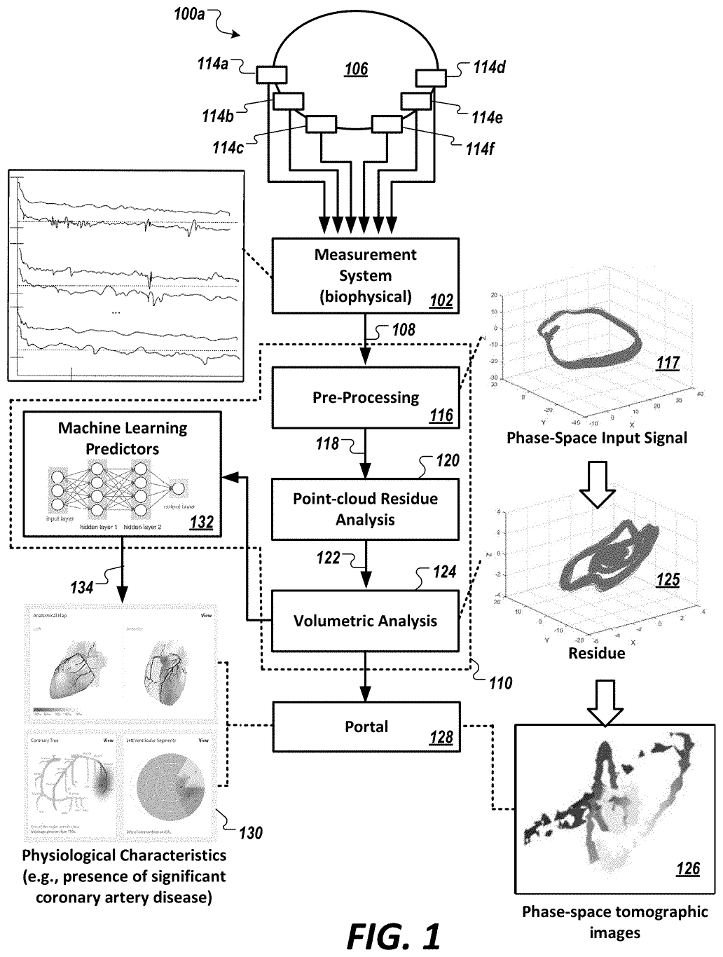

FIG. 1 is a diagram of an exemplary system 100 (shown as 100a) configured to generate a residue model to assess (e.g., non-invasively assess) a physiological system to predict and/or estimate presence or non-presence of disease in such physiological system, in accordance with an illustrative embodiment. As noted herein, physiological systems can refer to the cardiovascular system, the pulmonary system, the renal system, the nervous system, and other functional systems and sub-systems of the body. In the context of the cardiovascular system, the particular embodiment of system 100 shown in FIG. 1 facilitates the investigation of complex, nonlinear systems of the heart by examining in phase space the states, or phases, that such a system may exhibit over many cycles.

The system 100a generates a three-dimensional representation (or equivalent two-dimensional representation) of the residue model within a set of acquired biophysical signals collected by a measurement system 102 (also referred to as “phase space recorder” or “PSR” device). The term generally refers to a methodology that directly represent a physiological system, or sub-system of interest, as a multidimensional space in which each of the axes corresponds to one of the variables required to represent the state of the system. Residue model of other biophysical signal types (e.g., waveforms of photoplethysmographic signals) as discussed herein may be generated.

In FIG. 1, measurement system 102 is a non-invasive embodiment (shown as “Measurement System” 102) that acquires a plurality of biophysical signals via any number of measurement probes 114 (shown as probes 114a, 114b, 114c, 114d, 114e, and 114f) from a subject 106 to produce a biophysical-signal data set 108. The biophysical signal data set 108 includes a plurality of acquired signals (e.g., acquired from three distinct channels), which can be combined together to generate a multi-dimensional data set, e.g., a three-dimensional phase space representation, of the biophysical-signal data set 108. Measurement system 102 is configured to transmit, e.g., over a communication system and/or network, or via a direct connection, the acquired biophysical-signal data set 108, or a data set derived or processed therefrom, to a repository (e.g., a storage area network) (not shown) that is accessible by a non-invasive biophysical-signal assessment system 110 to be evaluated by an analytic engine executing an analysis of the acquired biophysical-signal data set 108 to determine a clinical output (includes an assessment of the presence or non-presence of a disease and/or an estimated physiological characteristic of the physiological system under study). In some embodiments, the clinical output includes an assessment of the presence or non-presence of a disease and/or an estimated physiological characteristic of the physiological system under study. In other embodiments, there is no clinical output but rather output of information that may be used by a clinician to provide their own clinical assessment of the information relative to the patient whose signals are being assessed.

As shown in FIG. 1, in some embodiments, non-invasive biophysical-signal assessment system 110 includes a Pre-Processing module 116 configured to perform signal processing operations on the acquired biophysical-signal data set 108 to generate a pre-processed biophysical signal data set 118 (e.g., a pre-processed phase gradient biophysical signal data set or a pre-processed wide-band phase gradient biophysical signal data set). In some embodiments, the analysis may be performed and interpreted in phase space. In some embodiments, the analysis may employ Poincare-based analysis. In some embodiments the analysis may be based on spectral analysis.

In some embodiments, the Pre-Processing module 116 is configured to perform a baseline wander removal operation (e.g., phase-linear baseline wander removal operation), a normalization operation (e.g., phase-linear signal normalization operation), and/or a down-sampling operation (e.g., phase-linear down-sampling operation). As used herein, the term “phase linear” generally refers to phase-neutral filters and operators that do not introduce any phase distortions into a signal, thereby preserving the phase information in the signal for subsequent analysis (e.g., phase space analysis). An example of the pre-processed biophysical signal data set 118 in phase space for a cardiac signal is shown in plot 117. The phase linear operation may be performed with respect to multiple channels of a same first signal type in conjunction with a second set of signals concurrently acquired with the first signal type. For example, acquisition between wide-band phase gradient cardiac signals may be performed with pre-defined phase with acquisition of waveforms of photoplethysmographic signals.

### Residue Model Example #1: Chaotic Analysis

In some embodiments, the biophysical-signal assessment system 110 as configured in FIG. 1 includes an analysis modeling module 120 (shown as “Decomposer” 120) configured to model, e.g., via a sparse-approximation signal decomposition algorithm, the quasi-periodic components of the pre-processed biophysical signal data set 118 to generate a residue data set in point cloud that models or estimates (122) the functional components of the physiological system. In some embodiments, the volumetric analysis module 124 facilitates the generation of a volumetric object of the point-cloud residue model. In some embodiments, the volumetric object corresponds to an isolation of the quasi-periodic, chaos, or spectral behavior(s) of the physiological system from other types of physiological behavior. The volumetric analysis module 124, in some embodiments, is configured to use the generated model 122 as a residue 125 (shown as “Residue” 125) of the pre-processed biophysical signal data set 118, e.g., subtracted by the model 122.

In some embodiments, the images 126 of volumetric object may be generated which can be colorized (as, for example, shown in FIG. 1), in some embodiments, with a second data set, e.g., derived based on an irrational fractional derivative operation performed on the pre-processed biophysical signal data set 118, the modelled biophysical signal data set 122, or the residue data set 125. The data set generated from the irrational fractional derivative operation, e.g., as expressed in color information, can provide a visually accessible assessment of rates of changes (e.g., a global rate of change) within the quasi-periodic operations of the physiological system (e.g., as compared to localized rate of change via a derivative operation). Such information is useful for a clinician when assessing the physiological state of the patient or subject whose biophysical signals are being assessed. Results of other analysis may be similar overlaid as a color data set of the three-dimensional volumetric object.

### Residue Model Example #2: Phase Analysis

In some embodiments, the residue model is generated from a residue subspace dataset determined by generating a first wavelet signal dataset by performing a first wavelet operation (via, e.g., a first phase linear wavelet operator) on data derived from the plurality of wide-band gradient signals; generating a second wavelet signal dataset by performing a second wavelet operation (via, e.g., a second phase linear wavelet operator) on the first wavelet signal data; and subtracting values of the first wavelet signal dataset from values of the second wavelet signal dataset to generate the residue subspace dataset, wherein the residue subspace dataset comprises a three-dimensional phase space dataset in a space-time domain.

Further description of such residue model is described in U.S. Pat. No. 10,362,950, which is incorporated by reference herein in its entirety.

Referring back to FIG. 1, system 100a, in some embodiments, includes a healthcare provider portal (shown as “Portal” 128) configured to display stored phase space analysis data sets/images 126 (among other intermediate data sets) in a phase space analysis report and/or angiographic-equivalent report. Portal 128, which in some embodiments may be termed a physician or clinician portal 128, is configured to access, retrieve, and/or display or present reports and/or the volumetric images 126 (and other data) for the report) from a repository (e.g., a storage area network).

In some embodiments, and as shown in FIG. 1, the healthcare provider portal 128 is configured to display the images 126 (or intermediate data set derived therefrom) in, or along with, an anatomical mapping report 130, a coronary tree report 130, and/or a 17-segment report 130. Portal 128 may present the data, e.g., in real-time (e.g., as a web object), as an electronic document, and/or in other standardized or non-standardized image visualization/medical data visualization/scientific data visualization formats. The physician or clinician portal 128, in some embodiments, is configured to access and retrieve reports or the phase space volumetric data sets/images (e.g., 130) (and other data) for the report) from a repository (e.g., a storage area network). The healthcare provider 128 and/or repository can be compliant with patient information and other personal data privacy laws and regulations (such as, e.g., the U.S. Health Insurance Portability and Accountability Act of 1996 and the EU General Data Protection Regulation) and laws relating to the marketing of medical devices (such as, e.g., the US Federal Food and Drug Act and the EU Medical Device Regulation). Further description of an example healthcare provider portal 128 is provided in U.S. Publication No. 2018/0078146, title “Method and System for Visualization of Heart Tissue at Risk”, which is incorporated by reference herein in its entirety. Although in certain embodiments, Portal 128 is configured for presentation of patient medical information to healthcare professionals, in other embodiments, the healthcare provider portal 128 can be made accessible to patients, researchers, academics, and/or other portal users.

The anatomical mapping report 130, in some embodiments, includes one or more depictions of a rotatable and optionally scalable three-dimensional anatomical map of cardiac regions of affected myocardium. The anatomical mapping report 130, in some embodiments, is configured to display and switch between a set of one or more three-dimensional views and/or a set of two-dimensional views of a model having identified regions of myocardium. The coronary tree report 130, in some embodiments, includes one or more two-dimensional view of the major coronary artery. The 17-segment report 130, in some embodiments, includes one or more two-dimensional 17-segment views of corresponding regions of myocardium. In each of the report, the value (e.g., 134) that indicates presence of cardiac disease or condition at a location in the myocardium, as well as a label indicating presence of cardiac disease (e.g., 134), may be rendered as both static and dynamic visualization elements that indicates area of predicted blockage, for example, with color highlights of a region of affected myocardium and with an animation sequence that highlight region of affected coronary arter(ies). In some embodiments, each of the report includes textual label to indicate presence or non-presence of cardiac disease (e.g., presence of significant coronary artery disease) as well as a textual label to indicate presence (i.e., location) of the cardiac disease in a given coronary artery disease.

In the context of cardiovascular systems, in some embodiments, the healthcare provider portal (and corresponding user interface) 128 is configured to present summary information visualizations of myocardial tissue that identifies myocardium at risk and/or coronary arteries that are blocked. The user interface can be a graphical user interface (“GUI”) with a touch- or pre-touch sensitive screen with input capability. The user interface can be used, for example, to direct diagnostics and treatment of a patient and/or to assess patients in a study. The visualizations, for a given report of a study, may include multiple depictions of a rotatable three-dimensional anatomical map of cardiac regions of affected myocardium, a corresponding two-dimensional view of the major coronary arteries, and a corresponding two-dimensional 17-segment view of the major coronary arteries to facilitate interpretation and assessment of architectural features of the myocardium for characterizing abnormalities in the heart and in cardiovascular functions.

Indeed, in some embodiments, the three-dimensional volumetric object generated from a residue analysis, and parameters derived therefrom, may be interpreted manually or used as part of a machine learned classifier or predictor module that may be configured to assist in the determination of the presence or absence of disease or condition. Such a module may be local or remote to the assessment system 110. In some embodiments, and as shown in FIG. 1, the system 100a includes a predictor module 132 (shown as “Machine Learning Predictors” 132) that is configured to generate indicators 134 of presence or absence of disease or conditions (e.g., binary indicator of disease present and/or binary indicator of disease present in specific regions of the physiological region), which can be co-presented on the report 130 via the healthcare provider portal 128. In the context of cardiovascular disease, and as shown in FIG. 1, the predictor module 132 is configured to generate indicators 134 of presence or absence of coronary artery disease (e.g., presence or absence of significant coronary artery disease) in specific regions of the myocardium and/or coronary arteries.

In an example, an analysis of the characteristics of a physiological system is performed from a set of acquired biophysical signals. In the context of the cardiovascular system, cardiac phase signals can exhibit complex nonlinear variability that may not be efficiently described by traditional modeling techniques. Without wishing to be bound to a particular theory, it is believed that in a nonlinear system such as the cardiovascular system there is a cascade effect whereby components of the system act upon and amplify other components. In the heart, the conduction system and the heart muscle itself may act upon/affect each other—and in turn affect and are affected by the vasculature. In a chaotic system, small changes in initial conditions may be amplified in the same or similar manner, resulting in behavior that seems (e.g., to a person) unfathomably complex. That is, it may appear to be random. As described in V. Sharma, “Deterministic Chaos and Fractal Complexity in the Dynamics of Cardiovascular Behavior: Perspectives on a New Frontier,” Open Cardiovasc Med J., pp 110-12 3 (September 2009), the observed chaotic component may not be truly random, and can include encoding of a type of biological memory, which can allow the physiological system to revisit previous states without requiring that these states be directly encoded or repetitious. In contrast, truly random behavior contains no such biological memory or state and is instead associated with a decline in system, and hence biological, health. Without wishing to be bound to a particular theory, it is believed that a decline in chaotic behavior may diminish the ability of a given physiological system to adapt to a stimulus subjected to the system, thereby causing it to become periodic, which can be attributed to being deleterious to health. Similar investigations and observations have been made about the brain and other physiological systems. As later discussed, it can be readily observed that neurological signal data sets similarly contain both quasi-periodic signals and chaotic signals that can be analyzed using the exemplary analysis.

Three-dimensional volumetric objects may be used as a functional representation of a residue analysis of the physiological system.

In some embodiments, an analysis entails first modelling components of the acquired signals to a model of the signals and then subtracting the modelled signal data set from the acquired biophysical signal data set to determine a residue point cloud as the functional representation of the characteristics of the physiological system. The residue point-cloud model remaining, say, once the modelled signal has been subtracted from the input signal contains none of the traditional landmarks of the acquired biophysical signal (for a cardiac biophysical signal, the residue contains none of the traditional landmarks, say, as observed in conventional electrocardiogram (ECG) trace, which by its nature is a quasi-periodic signal).

When the point-cloud residue is presented as a volumetric object, features sets derived from the object can be used readily in a classification or a machine learned operation to estimate/predict and display contextual information on a patient's health, including the status of specific physiologic system health (e.g., cardiac health, a brain/neurological health, pulmonary health, and other biological system health). Volumetric object of residue point cloud data set can be synthesized and displayed via shapes and colors to represent the electrical and/or functional behavior and/or characteristics of the heart or other physiological systems.

Volumetric Object of a Residue Point-Cloud Model of an Analysis of a Physiological System

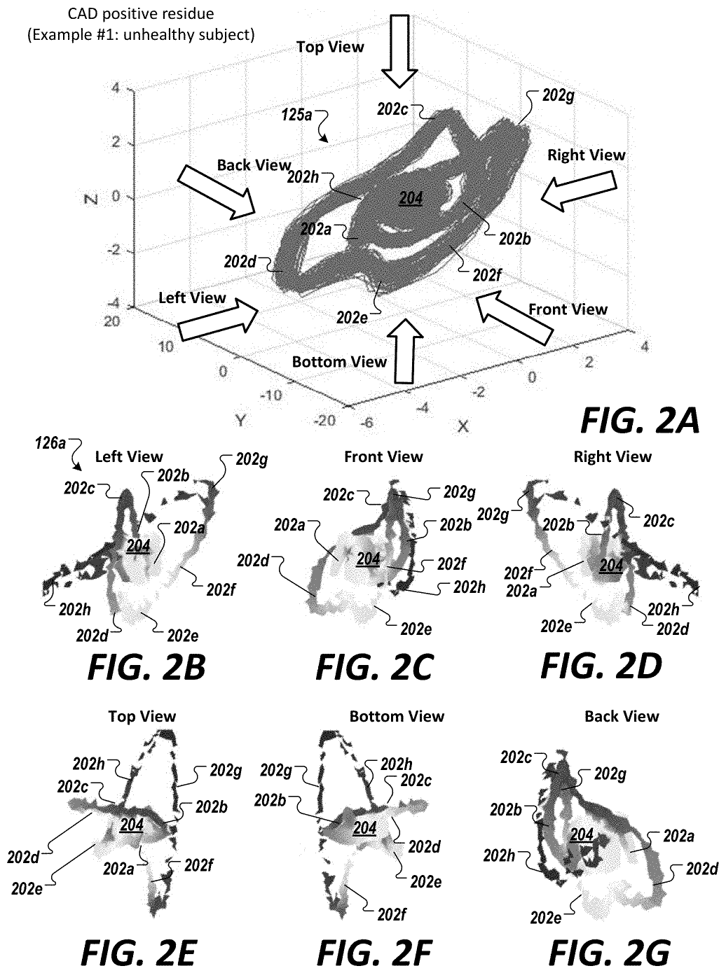

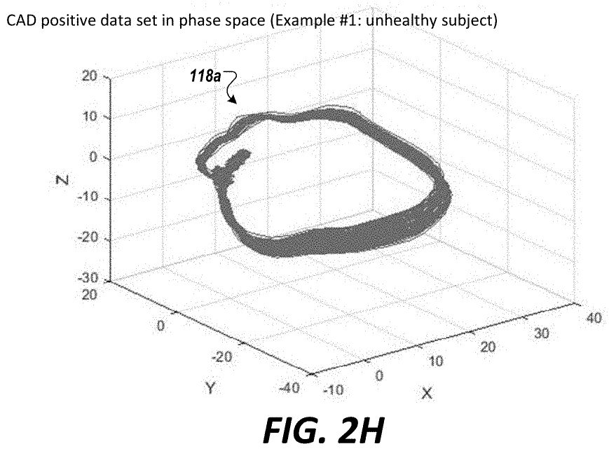

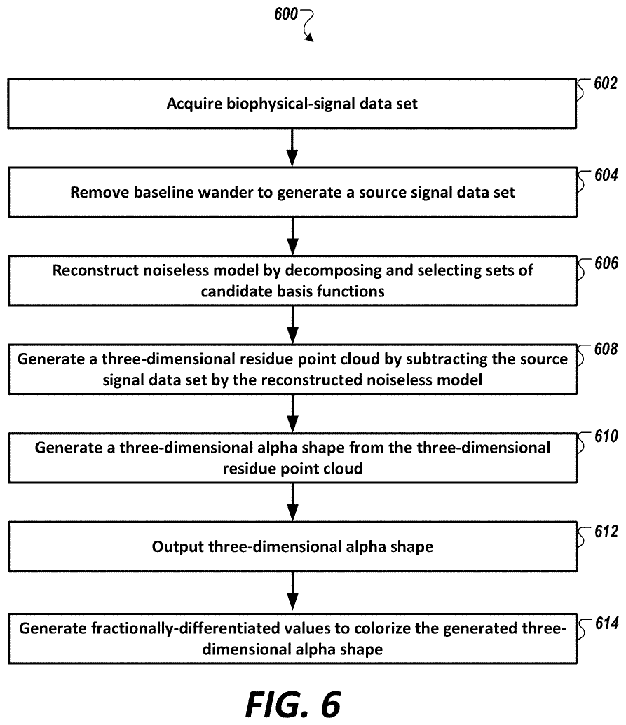

FIG. 2A shows a plot of an example volumetric object derived from residue point-cloud model/data set 125 (shown as 125a) generated from an analysis of a cardiac system in which the residue point-cloud model/data set has a positive diagnosis (i.e., unhealthy assessment) for presence of significant coronary artery disease, in accordance with an illustrative embodiment. As shown in FIG. 2A, the volumetric object of the residue point-cloud model of a CAD-positive patient is observed to have a more restrictive set of possible deterministic states as evidenced by geometric and tomographic features relating to observable loops in the residue data set 125. FIGS. 2B, 2C, 2D, 2E, 2F, 2G are example images 126 (shown as 126a) as different projected views of the volumetric object of the residue point-cloud model/data set 125. Specifically, as a three-dimensional phase space volumetric alpha shape object (α=0.55) generated from the residue point-cloud model/data set 125a of FIG. 2A, in accordance with an illustrative embodiment. Further description of a method to generate a three-dimensional volumetric object from the residue point-cloud model/data set is provided in the description provided in relation to FIG. 6.

Per FIGS. 2A-2G, it can be observed that the residue point-cloud model/data set 125a for a CAD-positive subject includes a number of connected loops that are large, distinct and off-axis (or orthogonal) to one another. Without wishing to be bound to a particular theory, it is believed that these geometric and tomographic features relating to observable loops in the residue point-cloud model/data set are representation of a more restrictive set of possible states as isolated in the residue point-cloud model/data set 125. Corresponding markers 202a-202h are shown in between FIG. 2A (residue point-cloud model/data set) and each of FIGS. 2B-2G. FIG. 2H shows, in phase space, the pre-processed biophysical signal data set 118 (shown as 118a) that is used to generate the residue point-cloud model/data set 125a of FIG. 2A, in accordance with an illustrative embodiment. It is noted that FIGS. 2A, 2H, 3A, 3H, 4A, 4H, 5A, 5H, and 10 shows examples of residue point-cloud model/data set. A point cloud is a set of data points in space (e.g., as described in three axes in three-dimensional space or two axes for two-dimensional space). The representation of the data are such that the individual data points are not necessarily shown, but the underly data set are in a point-cloud.

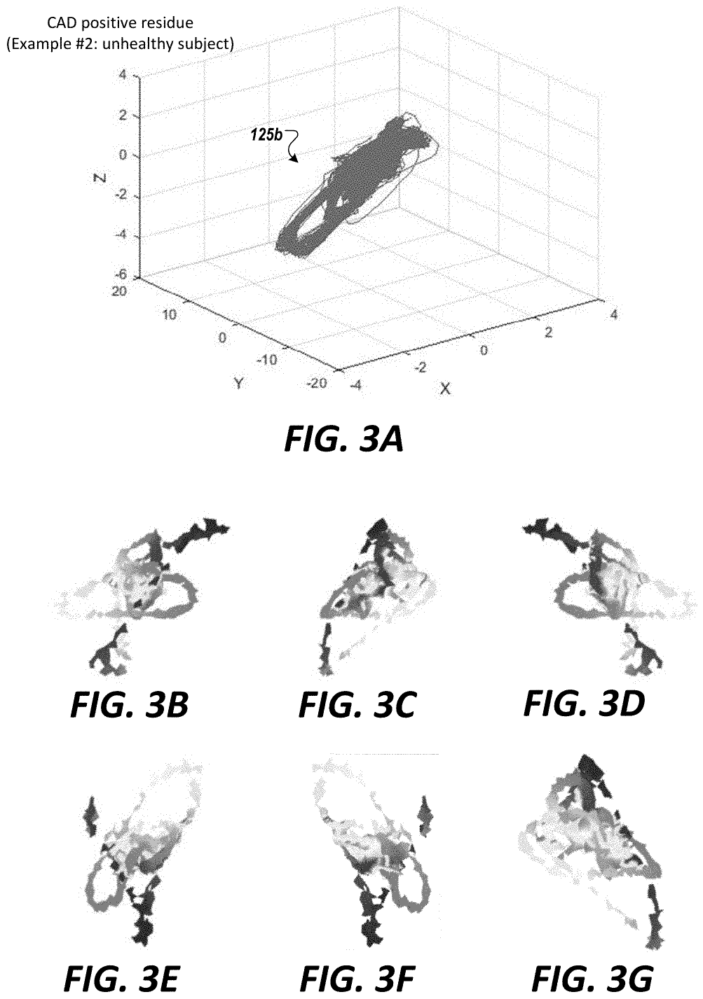

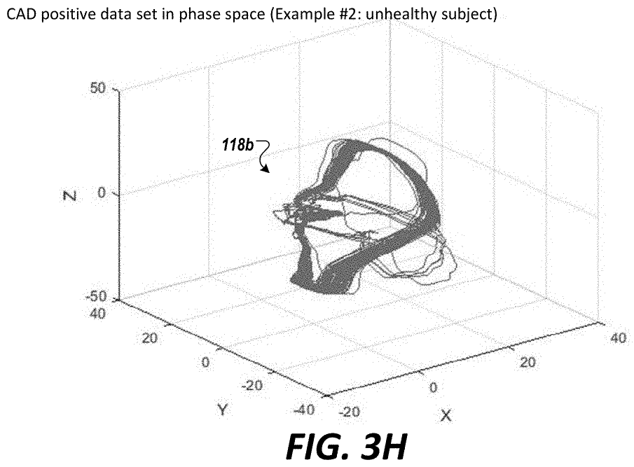

FIG. 3A shows a plot of another example residue point-cloud model/data set 125 (shown as 125b) representing the behavior of another cardiac system (of a different patient to that of FIG. 2A) that also has a positive diagnosis (i.e., unhealthy assessment) for presence of significant coronary artery disease, in accordance with an illustrative embodiment. FIGS. 3B, 3C, 3D, 3E, 3F, and 3G shows a volumetric object generated from the residue point-cloud model/data set 125b of FIG. 3A (of the second CAD-positive subject), in accordance with an illustrative embodiment. FIG. 3H shows, in phase space, the pre-processed biophysical signal data set 118 (shown as 118b) that is used to generate the residue model/data set 125a of FIG. 3A, in accordance with an illustrative embodiment.

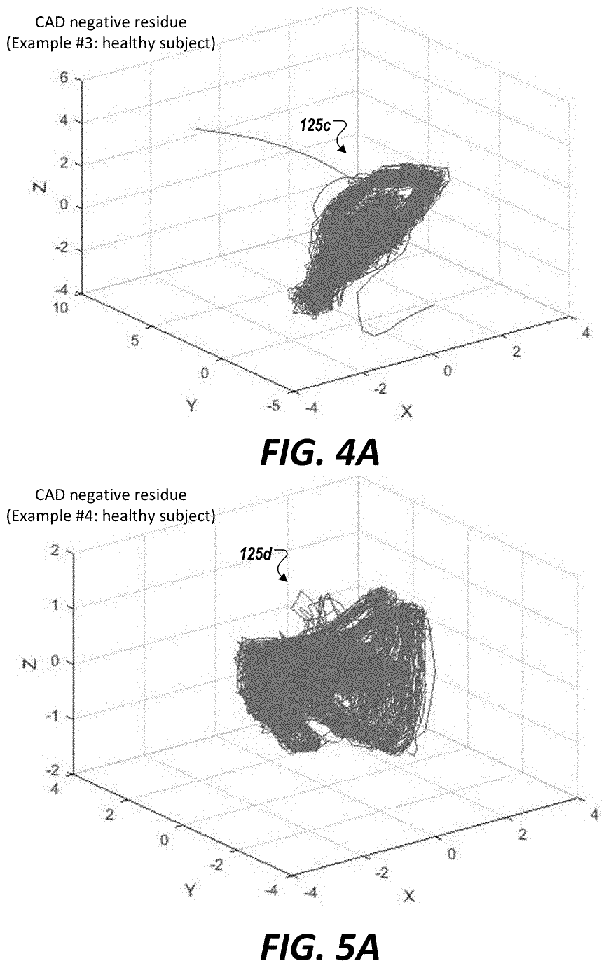

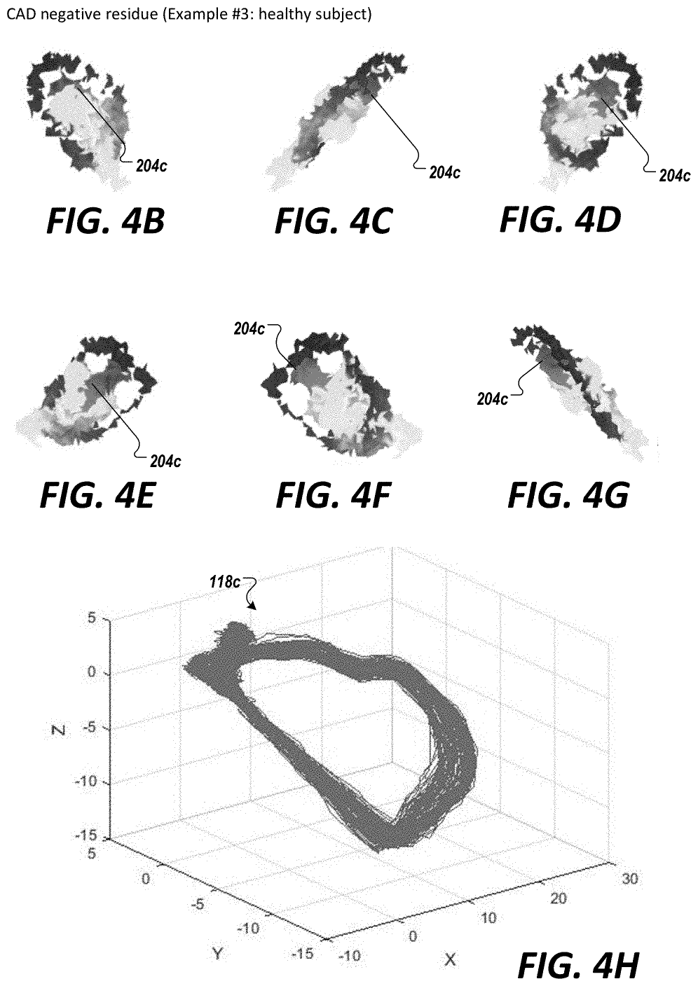

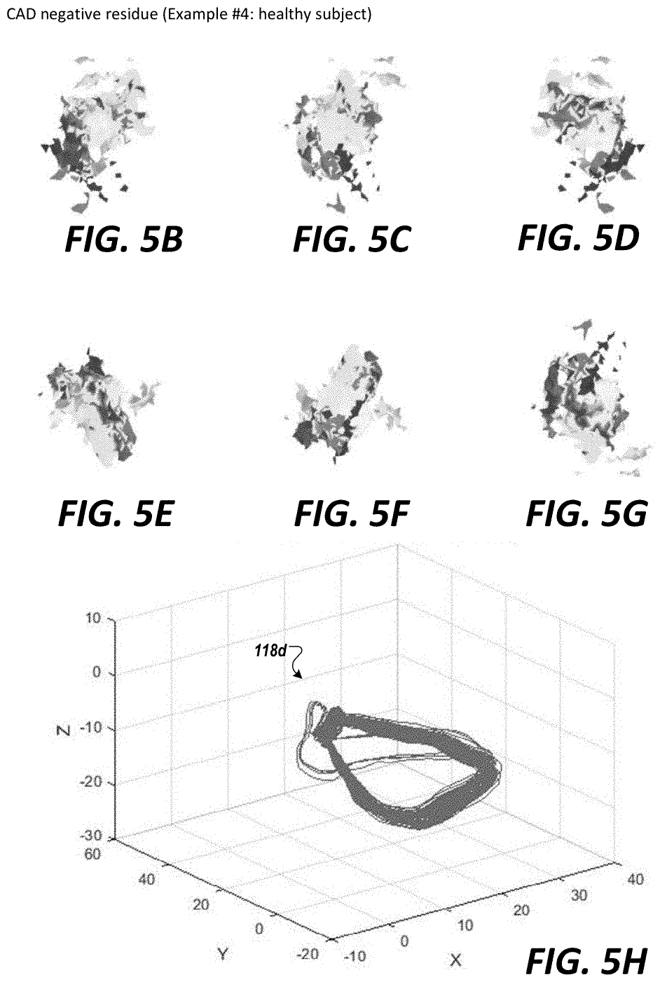

In contrast, FIGS. 4A and 5A each shows a plot of example residue point-cloud model/data set 125 (shown as 125c and 125d) representing the behavior of cardiac systems that have a negative diagnosis (e.g., a healthy assessment) for presence of significant coronary artery disease, in accordance with an illustrative embodiment. FIGS. 4B-4G and FIGS. 5B-5G show the corresponding three-dimensional volumetric object generated from the residue point-cloud model/data sets 125c and 125d of FIGS. 4A and 5A, respectively, in accordance with an illustrative embodiment. FIGS. 4H and 5H show, in phase space, the pre-processed biophysical signal data set 118 (shown as 118c and 118d) that is used to generate the residue point-cloud model/data set 125c and 125d of FIGS. 4A and 5A, respectively, in accordance with an illustrative embodiment.

Per FIGS. 4A-4G and 5A-5G, it can be observed that the volumetric object generated from residue point-cloud model/data sets 125c and 125d for a CAD-negative subject does not include interconnected loops as observed with respect to the CAD-positive data set. In addition, the color information associated with fractional derivative(s) of the signals appears to be different between the CAD-positive and CAD-negative data sets, with the CAD-negative exhibiting the highest fractional derivative values (shown as 204c and 204d in FIGS. 4B-4H and 5B-5H, respectively) concentrated around the center region of the volumetric object.

Example Method to Construct a Three-Dimensional Volumetric Object from a Point-Cloud Residue

FIG. 6 is a diagram of an exemplary method 600 of generating a three-dimensional volumetric object from a point-cloud residue of an analysis of a physiological system in accordance with an illustrative embodiment.

The method 600 includes acquiring at step 602 a biophysical data set, e.g., from the measurement system 102 or from a data repository having received the biophysical data set from the measurement system 102, e.g., as described in relation to FIG. 1. The measurement system 102, in some embodiments, is configured to acquire biophysical signals that may be based on the body's biopotential via biopotential sensing circuitries as biopotential biophysical signals. In the cardiac and/or electrocardiography contexts, measurement system 102 is configured to capture cardiac-related biopotential or electrophysiological signals of a living organism (such as a human) as a biopotential cardiac signal data set. In some embodiments, measurement system 102 is configured to acquire a wide-band cardiac phase gradient signals as a biopotential signal or other signal types (e.g., a current signal, an impedance signal, a magnetic signal, an optical signal, an ultrasound or acoustic signal, etc.). The term “wide-band” in reference to an acquired signal, and its corresponding data set, refers to the signal having a frequency range that is substantially greater than the Nyquist sampling rate of the highest dominant frequency of a physiological system of interest. For cardiac signals, which typically have dominant frequency components between about 0.5 Hz and about 80 Hz, the wide-band cardiac phase gradient signals or wide-band cardiac biophysical signals comprise cardiac frequency information at a frequency selected from the group consisting between about 0.1 Hz and about 1 KHz, between about 0.1 Hz and about 2 KHz, between about 0.1 Hz and about 3 KHz, between about 0.1 Hz and about 4 KHz, between about 0.1 Hz and about 5 KHz, between about 0.1 Hz and about 6 KHz, between about 0.1 Hz and about 7 KHz, between about 0.1 Hz and about 8 KHz, between about 0.1 Hz and about 9 KHz, between about 0.1 Hz and about 10 KHz, and between about 0.1 Hz and greater than 10 KHz (e.g., 0.1 Hz to 50 KHz or 0.1 Hz to 500 KHz). In addition to capturing the dominant frequency components, the wide-band acquisition also facilitate capture of other frequencies of interest. Examples of such frequencies of interest can include QRS frequency profiles (which can have frequency ranges up to about 250 Hz), among others. The term “phase gradient” in reference to an acquired signal, and its corresponding data set, refers to the signal being acquired at different vantage points of the body to observe phase information for a set of distinct events/functions of the physiological system of interest. Following the signal acquisition, the term “phase gradient” refers to the preservation of phase information via use of non-distorting signal processing and pre-processing hardware, software, and techniques (e.g., phase-linear filters and signal-processing operators and/or algorithms). In some embodiments, other signal types are acquired in combination with the biopotential biophysical signals, e.g., waveforms of photoplethysmographic signals, to which phase analysis, e.g., to generate point-cloud residue and three-dimensional volumetric object, between the multiply acquired signals may be performed.

In the neurological context, measurement system 102 is configured to capture neurological-related biopotential or electrophysiological signals of a living subject (such as a human) as a neurological biophysical signal data set. In some embodiments, measurement system 102 is configured to acquire wide-band neurological phase gradient signals as a biopotential signal or other signal types (e.g., a current signal, an impedance signal, a magnetic signal, an ultrasound, an optical signal, an ultrasound or acoustic signal, etc.). Examples of measurement system 102 are described in U.S. Publication No. 2017/0119272 and in U.S. Publication No. 2018/0249960, each of which is incorporated by reference herein in its entirety.

In some embodiments, measurement system 102 is configured to capture wide-band biopotential biophysical phase gradient signals as unfiltered electrophysiological signals such that the spectral component(s) of the signals are not altered. Indeed, in such embodiments, the wide-band biopotential biophysical phase gradient signals are captured, converted, and even analyzed without having been filtered (via, e.g., hardware circuitry and/or digital signal processing techniques, etc.) (e.g., prior to digitization) that otherwise can affect the phase linearity of the biophysical signal of interest. In some embodiments, the wide-band biopotential biophysical phase gradient signals are captured in microvolt or sub-microvolt resolutions that are at, or significantly below, the noise floor of conventional electrocardiographic, electroencephalographic, and other biophysical-signal acquisition instruments. In some embodiments, the wide-band biopotential biophysical signals are simultaneously sampled having a temporal skew or “lag” of less than about 1 microseconds, and in other embodiments, having a temporal skew or lag of not more than about 10 femtoseconds. Notably, the exemplified system minimizes non-linear distortions (e.g., those that can be introduced via certain filters) in the acquired wide-band phase gradient signal to not affect the information therein.

In some embodiments, six simultaneously sampled signals are captured from a resting subject as a raw differential channel signal data set (e.g., comprising channels that may be called “ORTH1”, “ORTH2”, and “ORTH3”) in which the signals embed the inter-lead timing and phase information of the acquired signals specific to the subject. Geometrical contrast arising from the interference in the phase plane of the depolarization wave with the other orthogonal leads can be used to facilitate superimposition of phase space information on a three-dimensional representation of, in one example, the heart. Noiseless subspaces further facilitate the observation of the phase of these waves. That is, the phase of the orthogonal leads carries the information about the structure and generates geometrical contrast in the image. Phase-contrast takes advantage of the fact that different bioelectric structures have different impedances, and so spectral and non-spectral conduction delays and bends the trajectory of phase space orbit through the heart by different amounts. In the cardiovascular context, these small changes in trajectory can be normalized and quantified beat-to-beat and corrected for abnormal or poor lead placement, and the normalized phase space integrals can be mapped to a geometric mesh for visualization.

In some embodiments, the non-invasive measurement system 102 is configured to sample a biophysical signal (e.g., bipolar biopotential signals) at about a sampling rate greater than about 1 kHz (e.g., about 8 kHz) for each of three differential channels orthogonally placed on a subject for a duration between about 30 and about 1400 seconds, e.g., for about 210 seconds. Other durations and sampling rates may be used.

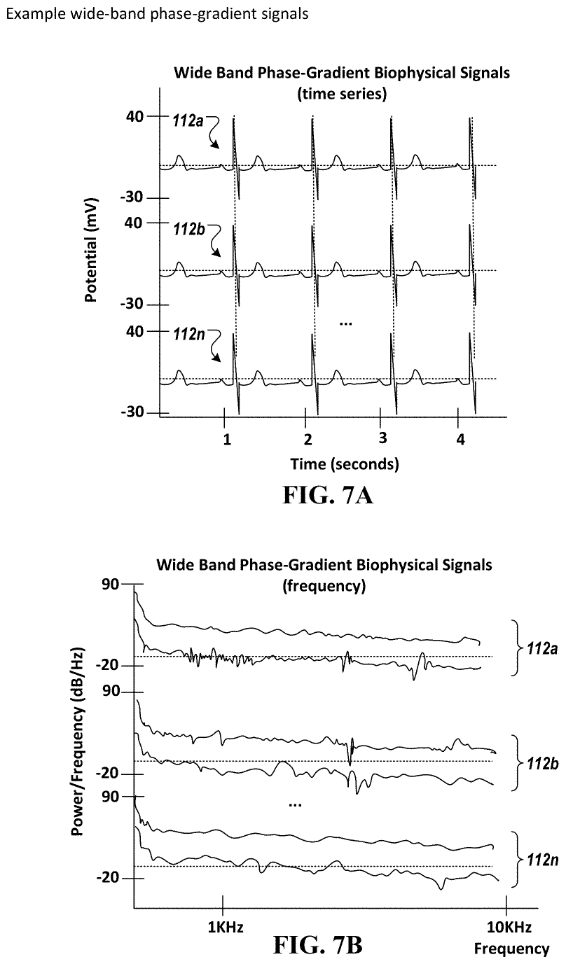

FIG. 7A is a diagram of a set of time-series plots of an example acquired cardiac signal data set 108 acquired from the non-invasive measurement system 102 of FIG. 1, in accordance with an illustrative embodiment. The plot shows the cardiac biophysical signal being acquired across multiple channels. The plot shows the data set being expressed in mV units over time (in seconds). Preferably, the wide-band phase-gradient biophysical signals are acquired from probes are placed on the body at orthogonal locations from one another.

FIG. 7B is a diagram of the example cardiac signal data set (e.g., wide-band phase gradient cardiac signal data set) of FIG. 7A shown in the frequency domain, in accordance with an illustrative embodiment. As shown in FIG. 7B, the cardiac signal data set has a frequency component greater than 1 kHz, which is significantly higher than conventional electrocardiogram measurements.

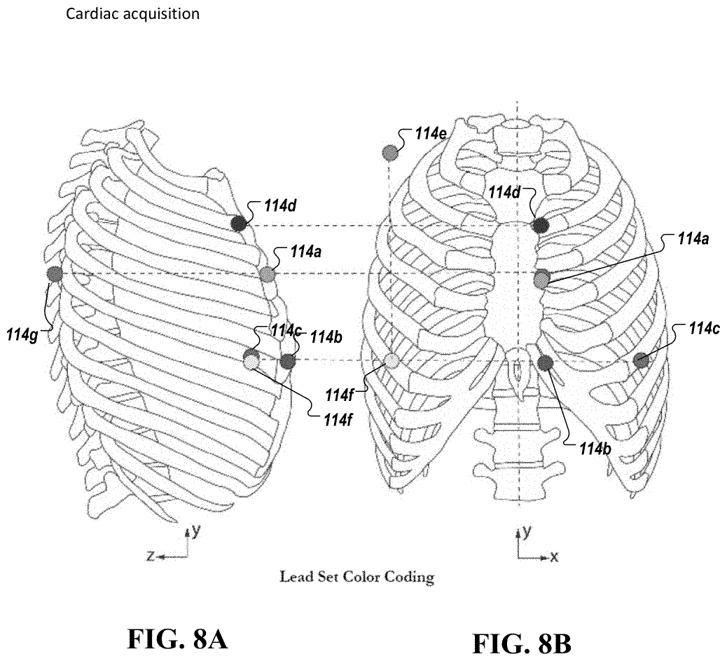

FIGS. 8A and 8B are diagrams showing an example placement of surface electrodes as probes 114a-114f at the chest and back of a patient or subject to acquire bio-potential signals associated with cardiac signal data set, in accordance with an illustrative embodiment. FIG. 8A shows a side view of placement of the surface electrodes 114a-114g to the chest and back of the patient, in accordance with an illustrative embodiment. FIG. 8B shows a front view of placement of the surface electrodes 106a-106g to the same, in accordance with an illustrative embodiment. As shown, the surface electrodes are positioned at (i) a first location proximal to a right anterior axillary line of the subject corresponding to a 5th intercostal space; (ii) a second location proximal to a left anterior axillary line corresponding to the 5th intercostal space; (iii) a third location proximal to a left sternal border corresponding to a 1st intercostal space; (iv) a fourth location proximal to the left sternal border below the sternum and lateral to a xiphoid process; (v) a fifth location proximal to the left sternal border corresponding to a 3rd intercostal space; (vi) a sixth location proximal to a back directly opposite of the fifth location and left of the patient's spine; and (viii) a seventh location proximal to a right upper quadrant of the patient corresponding to a 2nd intercostal space along a left axillary line.

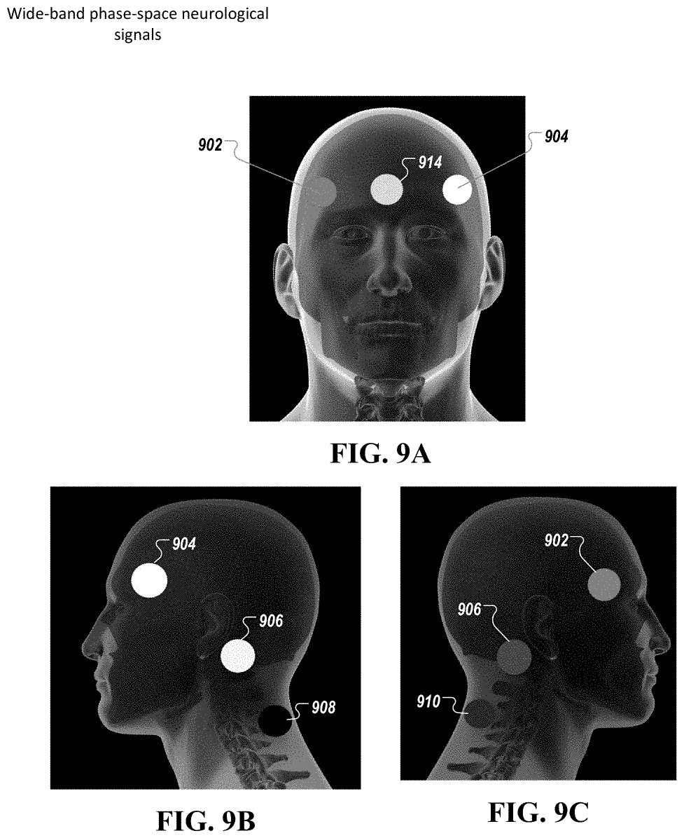

In addition to acquisition of a cardiac signal data set, the exemplified system 100a may be used to acquire neurological signal data sets (e.g., “wide-band phase-gradient cerebral signal data sets”). FIGS. 9A, 9B, and 9C are diagrams of an example placement of the surface electrodes at the head and neck of a patient or subject to acquire biopotential signals associated with neurological signal data set, in accordance with an illustrative embodiment. FIG. 9A shows a front view of placement of the surface electrodes to the patient. FIG. 9B and FIG. 9C each shows side views of placement of the surface electrodes to the same. As shown, a first set of two surface electrodes (shown as 902 and 904) corresponding to a first differential channel is placed at the left and right temple of the patient, a second set of two surface electrodes (shown as 906 and 908) corresponding to a second differential channel is placed under each ear of the subject, and a third set of two surface electrodes (shown as 910 and 912) corresponding to a third differential channel is placed at the back of each side of the subject's neck. A seventh surface electrode (shown as 914) corresponding to common-mode potential output of the measurement system 102 is shown placed at the center of the patient's forehead.

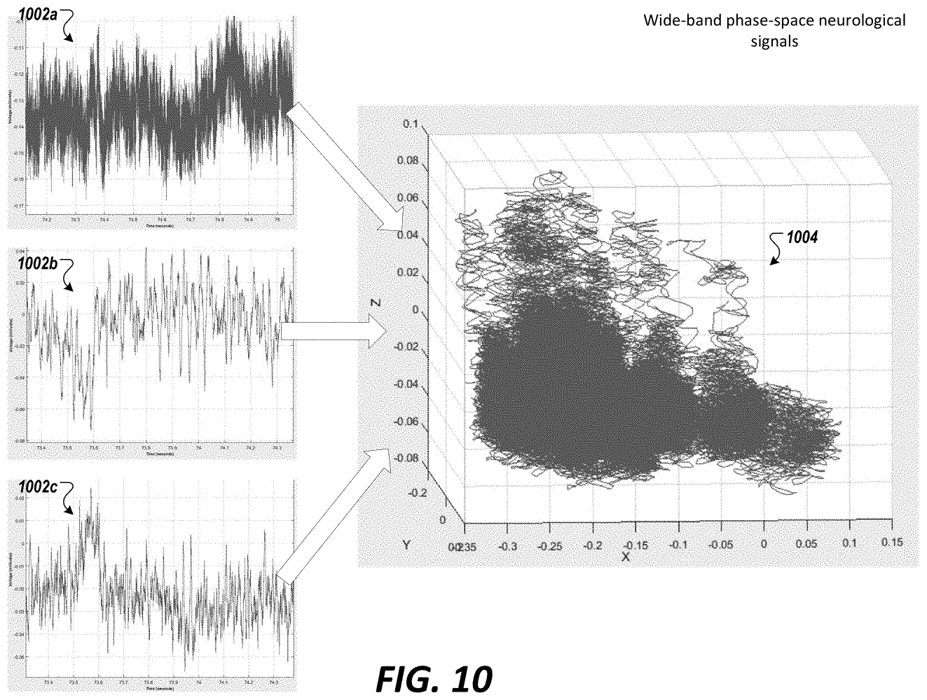

FIG. 10 is a diagram showing an example of an acquired neurological signal data set (shown as 1002a, 1002b, and 1002c) and its corresponding representation (shown as 1004) in phase space, in accordance with an illustrative embodiment. The neurological signal data set is generated from biopotential signals acquired from three differential channels (e.g., 1002a, 1002b, and 1002c). In FIG. 10, each of the data sets from channels 1002a, 1002b, 1002c is shown in a time-series plot with time is (x-axis) in which the amplitude is a measured voltage of the signal in millivolts (y-axis).

The phase space representation 1004 of the neurological signal data sets 1002a, 1002b, 1002c presents in each of the axes (shown as “X”, “Y”, and “Z”) the corresponding wide-band phase-gradient neurological signal data sets 1002a, 1002b, 1002c. It can be readily observed that the neurological signal data sets contain both quasi-periodic signals and chaotic signals and thus can be analyzed using techniques as disclosed herein that facilitate the modeling and analysis of quasi-periodic and chaotic signals for wide-band phase-gradient cardiac signals.

Referring to FIG. 6, method 600 further includes removing at step 604, by a processor (e.g., via the pre-processing module 116), a determined baseline wander from the raw differential channel signal of an acquired biophysical signal data set 108.

In the context of a cardiac signal, the baseline wander removal operation 602 is implemented, in some embodiments, as a phase-linear 2^{nd} order high-pass filter (e.g., a second-order forward-reverse high-pass filter having a cut-off frequency at 0.67 Hz). The forward and reverse operation ensures that the resulting pre-processed biophysical-signal data set 118 is phase-linear. Other phase-linear operations be used—e.g., based on wavelet filters, etc.

In other embodiments, a multi-stage moving average filter (median filter, e.g., with an order of 1500 milliseconds, smoothed with a 1-Hz low-pass filter) is used to extract a bias signal from each of the input raw differential channel signals. The bias is then removed from the signals by subtracting estimations of the signals using maximums of probability densities calculated with a kernel smoothing function.

In some embodiments, the signal is run though a signal quality test where the relevant output is the time-indices of the signal appropriate (of sufficient quality) for analysis. An example of the signal-quality test is described in U.S. Provisional Appl. No. 62/784,962, titled “Method and System for Automated Quantification of Signal Quality,” which is filed on Dec. 26, 2018, and incorporated by reference herein in its entirety.

In some embodiments, the method 600 further includes down-sampling the input signal or the pre-processed signal (e.g., to 1 kHz). In some embodiments, the down-sampling operation is an averaging operator or a decimation operator.

In some embodiments, the method further includes normalizing the input acquired biophysical signal data set 108 or the pre-processed signal 118. Similar types of down-sampling, baseline wander, and/or normalization operation can be applied to other biophysical-signal data sets.

In some embodiments, the method includes using only a portion of the acquired biophysical signal data set, e.g., that portion acquired after or before a pre-defined time or data set offset (e.g., after the first 31 seconds). It is observed, in some embodiments, that such operations can minimize and/or reduce motion artifacts (and therefore improve signal quality) that can be introduced by movement of a subject during the start of a measurement acquisition, therefore resulting in improved signal quality. It is also observed that such operations can minimize and/or reduce distortions (and therefore improve signal quality) in the measurement that can be attributed to probe placement and contacts and which is generally observed to reduce over the course of the measurement acquisition as the probe settles, also resulting in improved signal quality. Other time or data set offset techniques can be used; e.g., those based on quantification of noise in the acquired biophysical data set which may be the result of or associated with the biophysical signal acquisition protocol (instructions), types of probes or electrodes used, and the types and/or configurations of components such as cables for the transmission of signals, the biophysical signal measurement system, the biophysical signal acquisition space/environment, proximity to other medical equipment, etc.

Method 600 next includes at step 606 reconstructing a residue point cloud model. In some embodiments, the method decomposes the pre-processed biophysical-signal data set 118 into a linear combination of a set of selected candidate basis functions. As noted above, to isolate the deterministic behavior of the physiological system from other types of physiological behavior, a residue point cloud model/data set 125 is generated, e.g., by subtracting the pre-processed biophysical-signal data set 118, e.g., with the noiseless model. As discussed above, other techniques of generating a residue point cloud model may be employed.

In some embodiments, the modeling module has a greater than 99% accuracy. In some embodiments, the modeling module has a greater than 99.9% accuracy. In some embodiments, the modeling module has a greater than 99.99% accuracy. In some embodiments, the modeling module has a greater than 99.999% accuracy. In some embodiments, the modeling module has a greater than 99.9999% accuracy.

In some embodiments, a sparse-approximation signal decomposition algorithm is used that is configured to iteratively and recursively select candidate basis functions to add to the model based on a multi-dimensional (e.g., three dimensions) assessment of mutual information from all acquired channels (e.g., channels ORTH1, ORTH2, and ORTH3). That is, one or more terms (e.g., sine terms, cosine terms, complex exponential terms, etc.) are selected, by the processor, at each given iteration of the decomposer algorithm that reduce the collective error (e.g., mean-square error) between the model and all of the input signals (rather than for one time-series dimension). It is observed that this modeling technique can provide a model having a high degree of modeling accuracy suitable to accurately isolate the residue data set representing the deterministic chao behavior and/or characteristic of the physiological system. In some embodiments, the operation has also been observed to improve performance (e.g., processing time, longer duration models, etc.).

A sparse approximation modeling operation, as a mathematical reconstruction of the wide-band phase-gradient biophysical-signal data signal, is configured to determine, in some embodiments, a linear combination of candidate terms that are iteratively selected to approximate a source signal that is, or derived from, the biophysical-signal data set 108. In some embodiments, the sparse-approximation signal decomposition algorithm generates a model as a function with a weighted sum of basis functions in which basis function terms are sequentially appends to an initially empty basis to approximate a target function while reducing the approximation error.

In some embodiments, the sparse-approximation signal decomposition algorithm is configured to select complex exponential candidate terms and deriving pairs of sine and cosine terms (each with its own weight coefficients) from each selected complex exponential candidate term to add to the model. In some embodiments, the operation has been observed to further improve modeling performance (e.g., processing time, longer duration models, etc.).

In some embodiments, the sparse-approximation signal decomposition algorithm is configured to evaluate the addition of a new set of candidate basis functions without use of a nested loop (e.g., employing a matrix multiplication operator that, e.g., includes a positive-definite matrix for any inner product operation, e.g., involving an orthogonal expansion coefficient to avoid having nest loops, thereby increasing the number of model terms). In some embodiments, certain sparse-approximation signal decomposition algorithms that employ nested loops can recursively grow in a N² relationship with each added candidate functions, which can unduly restrict the number of candidate functions that a model can select. In some embodiments, the operation has been observed to further improve modeling performance (e.g., processing time, longer duration models, etc.).

In some embodiments, the sparse-approximation signal decomposition algorithm is configured to iteratively and recursively select candidate basis functions to add to the model until a stopping condition is reached (e.g., an assessed accuracy value reaches a pre-defined accuracy value (e.g., X %), the model reaches a maximum allowable number of candidates, and/or the model has included all available candidates).

As noted above, the residue remaining once the modelled signal that has been subtracted from the input signal contains none of the traditional landmarks of a conventional ECG trace. Of particular relevance when assessing a physiological system such as the cardiovascular system, and in particular its cardiac function, is any change in the structural properties of the heart (which may be induced or affected by pathology and/or aging) that can affect the heart's ability to respond to stimulation. For example, if the heart is less compliant due to remodeling, a decrease in myocyte membrane function, or a loss of cardiomyocytes, the mechanical function of the heart could be constrained and forced toward more periodic behavior.

In other embodiments, the sparse-approximation signal decomposition algorithm can employ a forward step and reverse step (e.g., a two steps forward, one step back approach, etc.) decomposition feature to improve accuracy. In another embodiment, the fast orthogonal search with first term reselection can be used, e.g., as described in McGaughey et al., “Using the Fast Orthogonal Search with First Term Reselection to Find Subharmonic Terms in Spectral Analysis,” Annals of Biomedical Eng., Vol. 31, issue 6, pp 741-751 (June 2003), which is incorporated by reference herein.

Indeed, the combination of these decomposition techniques can facilitate the selection of non-trivial phases of different frequencies in modeling the biophysical signal data set. In some embodiments, the modeling or analysis module 120 is configured to model the pre-processed biophysical signal data set 118 for over 6000 data points, e.g., for a duration of greater than 60 seconds at 1 kHz sampling.

In some embodiments, module 120 is configured to select candidate terms having frequency components ranging between about 0.1 Hz and about 10.0 Hz at about 0.1 Hz increments. In some embodiments, module 120 is configured to select candidate terms having frequency components ranging between about 0.1 Hz and about 20.0 Hz at about 0.1 Hz increments. In some embodiments, module 120 is configured to select candidate terms having frequency components ranging between about 0.1 Hz and about 30.0 Hz at about 0.1 Hz increments. In some embodiments, module 120 is configured to select candidate terms having frequency components ranging between about 0.1 Hz and about 40.0 Hz at about 0.1 Hz increments. In some embodiments, module 120 is configured to select candidate terms having frequency components ranging between about 0.1 Hz and about 50.0 Hz at about 0.1 Hz increments. In some embodiments, module 120 is configured to select candidate terms having frequency components ranging between about 0.1 Hz and greater than about 50.0 Hz at about 0.1 Hz increments.

In some embodiments, module 120 is configured to select candidate terms having frequency components ranging between about 0.01 Hz and about 10 Hz at about 0.01 Hz increments. In some embodiments, module 120 is configured to select candidate terms having frequency components ranging between about 0.01 Hz and about 20.00 Hz at about 0.01 Hz increments. In some embodiments, module 120 is configured to select candidate terms having frequency components ranging between about 0.01 Hz and about 30.00 Hz at about 0.01 Hz increments. In some embodiments, module 120 is configured to select candidate terms having frequency components ranging between about 0.01 Hz and about 40.00 Hz at about 0.01 Hz increments. In some embodiments, module 120 is configured to select candidate terms having frequency components ranging between about 0.01 Hz and about 50.00 Hz at about 0.01 Hz increments. In some embodiments, module 120 is configured to select candidate terms having frequency components ranging between about 0.01 Hz and greater than about 50.00 Hz at about 0.01 Hz increments.

Indeed, other decomposition algorithms that can provide model accuracy greater than about 99% can be used. As discussed in U.S. Publication No. 2013/0096394, which is incorporated by reference herein in its entirety, a sparse approximation operation comprises a set of operations, often iterative, to find a best matching projection of a data set (e.g., multi-dimensional data) onto candidate functions in a dictionary. Each dictionary can be a family of waveforms that is used to decompose the input data set. The candidate functions, in some embodiments, are linearly combined to form a sparse representation of the input data set. These operations can be numerical or analytical. In some embodiments, the mathematical reconstruction is based on evolvable mathematical models, symbolic regression, principal component analysis (PCA), matching pursuit, orthogonal matching pursuit, orthogonal search, linear models optimized using cyclical coordinate descent, projection pursuit, LASSO, fast orthogonal search, Sparse Karhunen-Loeve Transform, or combinations thereof. The recited examples are not exhaustive and other sparse approximation algorithms or methods may be used as well as any variations and/or combinations thereof.

Referring still to FIG. 6, the method 600 then includes at step 608 subtracting the model from the source signal to generate the residue point cloud model/data set 125. In some embodiments, the residue point cloud model/data set 125 is represented as a three-dimensional point cloud. The three-dimensional point cloud, in some embodiments, comprises a plurality of vertices and a plurality of faces defined by the plurality of vertices. In some embodiments, the source signal is the pre-processed biophysical-signal data set that has been down-sampled.

In some embodiments, the assessment system 110 is configured to generate grid coordinates from the model. The values of the grid coordinates may be directly subtracted from the source signal (e.g., the down-sampled pre-processed biophysical-signal data set).

In some embodiments, the analysis of the plurality of biophysical signal data sets comprises a quasi-periodic analysis of the frequency components of the plurality of biophysical signal data sets.

In some embodiments, the analysis of the plurality of biophysical signal data sets comprises a chaotic analysis of the frequency components of the plurality of biophysical signal data sets.

In some embodiments, the analysis of the plurality of biophysical signal data sets comprises a phase analysis of the plurality of biophysical signal data sets.

The method 600 then includes generating at step 610 a three-dimensional alpha shape from the three-dimensional residue point cloud data set. In some embodiments, the alpha parameter (namely; the radius of the sphere used to define adjacency) is set to about 0.55. In other embodiments, the alpha parameter may take on a different value. In some embodiments, assessment system 110 generates a first subspace “A” as the built signal from analysis and a second subspace “B” as the generated residue data set 125. The assessment system 110 is configured to, in some embodiments, combine subspace A and subspace C into a single point cloud using trihull code and alpha hull to generate a solid model as the phase space volumetric object. In other embodiments, Delaunay triangulation, alpha shapes, ball pivoting, Mesh generation, Convex Hull triangulation, and the like, is used. Table 1 shows pseudocode that can be used to generate a solid model from a point cloud data set, expressed as normalizedResidue matrix.

TABLE 1

alphaRadius = 0.55;
shp = alphaShape(normalizedResidue, alphaRadius);
triHullSubspace = boundaryFacets(shp);

Per Table 1, an alpha shape operator is performed on the normalizedResidue matrix to generate an alpha shape object. A boundaryFacets operator then acts on the alpha shape object to return a triangulation of the alpha shape (e.g., a matrix representing the facets that make up the boundary of the alpha shape in which the facets represent edge segments in 2-D and triangles in 3-D).

The generated three-dimensional alpha shape can be outputted at step 612 in a report in any number of useful formats. Other operations maybe used, including those described in relation to FIG. 1.

In some embodiments, method 600 then includes generating at step 614 fractionally-differentiated values to colorize, shade, gradate, and/or otherwise further differentiate aspects of the generated three-dimensional alpha shape to convey the information in a desired way. In some embodiments, the fractional derivative operation is based on Grunwald-Letnikov fractional derivative method. In some embodiments, the fractional derivative operation is based on Lubich's fractional linear multi-step method. In some embodiments, the fractional derivative operation is based on the fractional Adams-Moulton method. In some embodiments, the fractional derivative operation is based on the Riemann-Liouville fractional derivative method. In some embodiments, the fractional derivative operation is based on Riesz fractional derivative method. Other methods of performing a fractional derivative operation may be used.

In an embodiment, the system then colors the generated three-dimensional alpha shape by mapping each the triangular faces of the alpha shape to a color gradient, e.g., from blue to red, as an average of fractionally-differentiated values computed at three vertices of an analytical or numerical derivative of one of the input channels. Other embodiments may employ, in addition to or in substitution for the use of color/color gradients, non-color shading, gradation, and other techniques that can convey this information may be utilized and be within the scope of the present disclosure. Other report and reporting mechanisms maybe employed including those described in relation to FIG. 1.

In some embodiments, various views of the volumetric object generated from the residue point cloud model/data set are captured for presentation, e.g., via a secure web portal, to a healthcare provider (e.g., a physician) to assist the healthcare provider in the assessment of presence or non-presence of disease or condition (e.g., presence or non-presence of significant coronary artery disease). In some embodiments, the parametric features are derived from the volumetric object generated from the residue point cloud model/data set and are assessed by a trained neural network classifier configured to assess for presence or non-presence of a disease state or other condition (e.g., significant coronary artery disease). In some embodiments, the features are presented alongside the results of a machine-generated predictions to assist in the physician in making a diagnosis.

In some embodiments, system 100a generates one or more images 126 of the volumetric object generated from the residue point cloud model/data set in which the vertices, face triangulations, and vertex colors are presented. In some embodiments, multiple views of the representation is generated and included in a report (e.g., per FIGS. 2B-2G, etc.). In some embodiments, the one or more images are presented as a three-dimensional object that can be rotated, scaled, and/or panned based on user's inputs. Indeed, such presentation can be used to be assessed visually by a skilled operator to determine whether a subject has presence of non-presence of significant coronary artery disease. Non-visual modalities of presenting the same or similar information, such as providing data in tabular, summary, or even textual form (e.g., as written sentences or bullet points, etc.) may be used in some embodiments in varying configurations to accompany such images or even as a substitution for such images. Such modalities may also assist the operator in, e.g., interpreting images such as image 126, and may also provide additional information useful to the operator.

In variants in which the presentation of the volumetric object generated from the residue point cloud model/data set may be supplemented and/or enhanced to provide additional utility, the parametric features of the volumetric object generated from the residue point cloud model/data set are analyzed in machine learning operations (e.g., image-based machine learning operations or feature-based machine learning operations, e.g., via predictor module 132) to determine the one or more coronary physiological parameters (e.g., to aid a healthcare provider in making a diagnosis of disease). In some embodiments, the assessment system 110 (e.g., the predictor module 132) is configured to determine a volume metric (e.g., alpha hull volume) of the phase space analysis data set/image. In some embodiments, the assessment system 110 is configured to determine a number of distinct bodies (e.g., distinct volumes) of the generated phase space data set/image. In some embodiments, the assessment system 110 is configured to assess a maximal color variation (e.g., color gradient) of the generated volumetric object generated from the residue point cloud model/data set.

In some embodiments, the volumetric object generated from the residue point cloud model/data set, or parameters derived therefrom, may be used as part of a machine learned classifier or predictor module 132 (e.g., that is either local and/or remote to the assessment system 110) to assist in the determination of the presence or absence of disease. The predictor module 132 can generate indicators 134 of presence or absence of disease (e.g., binary indicator of disease present and/or binary indicator of disease present in specific regions of the physiological region), which can be co-presented on the report 130 via the physician or clinician portal 128.

In some embodiments, the machine-learned classifier is instantiated on a machine-learned classifier selected from the group consisting of GoogLeNets, ResNets, ResNeXts, DenseNets, and DualPathNets, e.g., to train a machine-learned classifier to predict presence or absence of coronary artery disease (and/or significant coronary artery disease). In some embodiments, the machine-learned classifier is instantiated on an artificial intelligence platform such as, e.g., IBM Watson, Microsoft Azure, Google Cloud AI, Amazon AI, etc. to train a custom machine-learned classifier to predict presence or absence of coronary artery disease or condition (and/or significant coronary artery disease).

The machine-learned classifier, in some embodiments, uses deep learning methods to classify images into one or more positive classes and/or one more negative classes. In some embodiments, the deep learning methods are used to train both a CAD-positive class and a CAD-negative class in which a positive class, in some embodiments, is one in which the class can be defined as being part of a membership and a negative class is one in which the class can be defined as an exclusion from any of the positive class membership.

For training purposes, three-dimensional volumetric objects of a residue point-cloud model from an analysis of a physiological system may be processed in a coronary artery disease automated assessment pipeline. The pipeline may retrieve cardiovascular-related wide-band phase-gradient biophysical signal data sets associated with training and validation as well as verification and gating functions as may be desired.

A machine learning algorithm (e.g., meta-genetic algorithm), in some embodiments, is used to generate predictors linking aspects of the phase model (e.g., pool of features) across a population of patients representing both positive (i.e., have a disease or condition) and negative (i.e., do not have a disease or condition) cases to detect the presence of myocardial tissue associated with, e.g., significant coronary artery disease. In some embodiments, the performances of the candidate predictors are evaluated through a verification process against a previously unseen pool of patients. In some embodiments, the machine learning algorithm invokes a meta-genetic algorithm to automatically select a subset of features drawn from a large pool of features. This feature subset can then be used by an algorithm such as an adaptive boosting (AdaBoost) algorithm to generate predictors to diagnose significant coronary artery disease across a population of patients representing both positive and negative cases. The performances of the candidate predictors are determined through verification against a previously unseen pool of patients. A further description of the AdaBoost algorithm is provided in Freund, Yoav, and Robert E. Schapire, “A decision-theoretic generalization of on-line learning and an application to boosting,” European conference on computational learning theory, Springer, Berlin, Heidelberg (1995), which is incorporated by reference herein in its entirety.

In other embodiments, predictor module 132 is configured to predict the presence or non-presence of significant coronary artery disease or condition from the three-dimensional volumetric object by projecting the object at pre-defined or even user-selectable set number of views (e.g., six views) (e.g., a top view, a bottom view, a front view, a back view, a left view, and a right view). In some embodiments, each projected image is first converted to grayscale and scaled to a pre-defined image resolution (e.g., less than 200×200 pixels). Other pixel count and image resolution values can be used. In some embodiments, the neural network classifier includes multiple hidden neurons (e.g., up to 15 hidden neurons) with leaky rectified linear activations. Dropout may be used between the hidden layer and the final output neuron to prevent overfitting. L1 and L2 regularization penalties may also be applied. A binary cross entropy may be used as a loss function. Optimization may be performed using the gradient-based Adam algorithm, among others.

Heat maps and contour plots, in some embodiments, are generated from the outputs of the neural network classifier on a given phase space analysis data set/image or from the computed phase space images themselves.

In some embodiments, a 4×4 moving window of white pixels (e.g., having a value of 1 in grayscale images) is swept over the entire image, with the neural network classifier being evaluated once for each window position and the output of the neural network being recorded. When a given pixel in the phase space analysis data set/image is covered by the moving window more than once (e.g., when the window is larger than a single pixel but moving one pixel at a time), each pixel in the heat map may have a value that is an average output of the neural network classifier when the corresponding pixel in the phase space analysis data set/image is covered by the window. Contour plots may be generated using the same data as the heat maps.

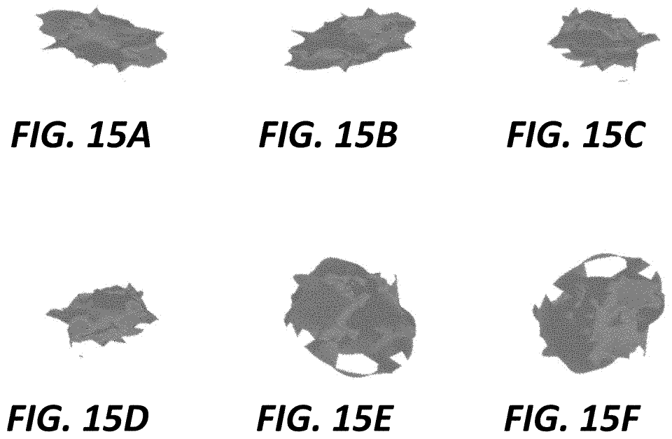

FIGS. 15A, 15B, 15C, 15D, 15E, and 15F shows show an example outputted classification for the presence and non-presence of significant coronary artery disease as determined via a neural network classifier, in accordance with an illustrative embodiment.

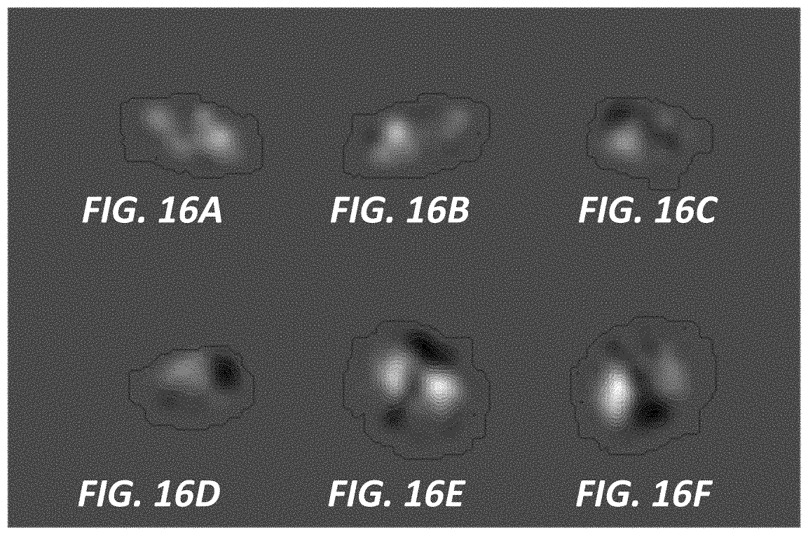

FIGS. 16A, 16B, 146C, 16D, 16E, and 16F show an example outputted classification overlaid with a contour data set and heat map associated with the classifier in accordance with an illustrative embodiment.

Example of heat maps and contour plots are further described in U.S. application Ser. No. 16/232,586, field on Dec. 26, 2018, title “Method and System to Assess Disease Using Phase Space Tomography and Machine Learning,” which is incorporated by reference.

Three-Dimensional Volumetric Object Interpretation Discussion

Without wishing to be bound to a particular theory, in one implementation of the volumetric object generated from the residue point cloud model/data set (e.g., in the assessment of chaotic or quasi-periodic behavior of the physiological system), CAD-positive data sets can be assumed to have a smaller set of disease etiologies (i.e., underlying causes) as compared to CAD-negative data sets. For example, chest pain as experienced by a CAD-negative patient can manifest from a larger set of etiologies. In contrast, chest pain as experienced by a CAD-positive patient may be more likely to be directly linked to, e.g., myocardial ischemia as induced by flow-limiting lesions associated with coronary artery disease. Indeed, the more restricted the possible available states observed in the chaotic behavior of the physiological system, the more likely the subject has an underlying disease or condition (associated with damaged tissue) that is responsible for the restriction, supporting a correlation to the geometric and topographic features observed in the phase space analysis data sets/images as disclosed herein.

In some embodiments, analysis of certain volumetric object generated from the residue point cloud model/data set may be represented visually with a repetitive set of distinct paths, or loops, in phase space (e.g., having two or more large distinct loops). CAD-positive data set, e.g., may have less fragmentation in the loops and high number of loops based on certain underlying analysis performed (e.g., based on quasi-periodic or chaotic analysis of the physiological system). CAD-positive data set are also expected to have orthogonal loops or at least have some angle between observed loops in such analysis. Such visual features can be observed in the plots of FIGS. 2A and 3A, which are shown to have high number of large and distinct loops (shown as 202a, 202b, 202c). In contrast, a CAD-negative data set as shown in the plots of FIGS. 4A and 5A might be expected not to have such large distinct loops, nor as many loops compared to those of a CAD-positive data set.

To make the three-dimensional point-cloud of the residue point cloud/model data set, e.g., of FIGS. 2A, 3A, 4A, and 5A more distinct for the identification of geometric and topologic features (e.g., loops), the analysis module 122, in some embodiments, is configured to transform the point-cloud data set into a solid volume (e.g., as shown in FIGS. 2B-2G, 3B-3G, 4B-4G, 5B-5G, etc.). In some embodiments, the analysis module 122 is configured to generate a three-dimensional alpha shape as a three-dimensional phase space volumetric object in which the alpha (the radius of the sphere used to define adjacency) is set to, e.g., 0.55. Other alpha values or solid volume generating algorithm (e.g., Delaney triangulation) can be used. The solid volume can more readily lend to geometric analysis to produce machine-extractable feature set that captures functional aspect of the physiological in expressing or characterizing a disease state.

FIGS. 2B, 2C, 2D, 2E, 2F, and 2G each shows different two-dimensional views (e.g., left view, front view, right view, top view, bottom view, and back view, respectively) of the three-dimensional volumetric object (α=0.55) generated from the residue point cloud model/data set 125a of FIG. 2A of a CAD-positive subject, in accordance with an illustrative embodiment.

FIGS. 3B, 3C, 3D, 3E, 3F, and 3G each shows different two-dimensional views (e.g., left view, front view, right view, top view, bottom view, and back view, respectively) of the three-dimensional volumetric object (α=0.55) generated from the residue point-cloud model/data set 125b of FIG. 3A of a CAD-positive subject, in accordance with an illustrative embodiment.

FIGS. 4C, 4D, 4E, 4F, 4G, and 4H each shows different two-dimensional views (e.g., left view, front view, right view, top view, bottom view, and back view, respectively) of the three-dimensional volumetric object (α=0.55) generated from the residue point-cloud model/data set 125c of FIG. 4A of a CAD-negative subject, in accordance with an illustrative embodiment.

FIGS. 5B, 5C, 5D, 5E, 5F, and 5G each shows different two-dimensional views (e.g., left view, front view, right view, top view, bottom view, and back view, respectively) of the three-dimensional volumetric object (α=0.55) generated from the residue point-cloud model/data set 125d of FIG. 5A of a CAD-negative subject, in accordance with an illustrative embodiment.

As shown in FIGS. 2B-2G, 3B-3G, 4B-4G, and 5B-5G, the triangular faces of the alpha shape can be colored with a second derived data set based on an irrational fractional derivative operation performed on the pre-processed wide-band phase-gradient biophysical signal data set 118, the modelled wide-band phase-gradient biophysical signal data set 122a, or the residue point-cloud model/data set 125 to represent the rate of global change of amplitude of the raw signal or of the model signal. As also shown in FIGS. 2B-2G, 3B-3G, 4B-4G, and 5B-5G, the analysis module 122 can map the second data set as a color gradient, from blue to red, that has a value calculated from an average derived from three neighboring vertices in which each vertex is the result of analytically-derived fractional derivative operation (e.g., having an irrational fractional order involving pi) performed on an acquired channel of the wide-band phase-gradient biophysical signal data set. The color therefore may represent the rate of global change of amplitude of the signal, with the blue color regions representing little change and the red color regions indicating the highest rate of global change.

Referring still to FIGS. 2B-2G, 3B-3G, 4B-4G, and 5B-5G, the red color regions of the presented coloring indicate rapidly changing amplitude, e.g., of the voltage gradient, at these points, and the blue color regions indicate relatively stable amplitudes.

Three-dimensional volumetric object of a residue point-cloud model (e.g., of quasi-periodic or chaos analysis), in some embodiments, can be expected to be represented visually with a repetitive set of distinct paths, or loops, in phase space (e.g., having two or more large distinct loops). One can also expect to observe that CAD-positive data set will have less fragmentation in the loops and high number of loops. One can also expect to observe that a CAD-positive data set will have orthogonal loops or at least have some angle between observed loops. Such visual features can be observed in FIGS. 2A and 3A, which are shown to have high number of large and distinct loops (shown as 202a, 202b, 202c). In contrast, a CAD-negative data set as shown in FIGS. 4A and 5A can be expected not to have large distinct loops nor many loops.

CAD-negative images (e.g., FIGS. 4B-4G and 5B-5G) appear to have concentrated, solid red cores while CAD-positive images (e.g., FIGS. 2B-2G and 3B-3G) appears to have loop-like structures. Without wishing to be bound by theory, it is believed that this observation indicates that CAD-negative subjects tend to show relatively unconstrained search spaces for deterministic chaos, particularly in areas such as depolarization, where rapidly changing voltage gradients could be observed. In contrast, CAD-positive images seem to have constrained, loop-like, chaos across the full range of cardiac function.

Indeed, a generated three-dimensional volumetric object of a residue model of a physiological system can be used to view the functional characteristic of that system.

### Experimental Results

A two-stage study called Coronary Artery Disease—Learning Algorithm Development (“CADLAD”) was implemented to support the development and testing of the machine-learned algorithms in connection with the present disclosure. In Stage 1 of the CADLAD study, paired human clinical biophysical data were used to guide the design and development of pre-processing, feature extraction, and machine learning phase of the development. That is, the collected clinical were split into three cohorts: training (50%), validation (25%), and verification (25%). Similar to the steps described above for processing signals from a patient for analysis, each signal was first pre-processed to clean and normalize the data. Following these processes, a set of features were extracted from the signals in which each set of features was paired with a representation of the true condition—for example, the binary classification of the presence or absence of significant CAD. The final output of this stage was a fixed algorithm embodied within a measurement system.

In Stage 2 of the CADLAD study, the machine-learned algorithms were used to provide a determination of significant CAD against a pool of previously untested clinical data. The criteria for disease for the CADLAD study was established as defined in the American College of Cardiology (ACC) clinical guidelines; specifically, greater than 70% stenosis as determined by angiography or less than a 0.80 fractional-flow reserve (“FFR”) value as measured by flow wire.

In an aspect of the CADLAD study, an assessment system was developed that automatically and iteratively explores combines features in various functional permutations with the aim of finding those combinations which can successfully match a prediction based on the features. To avoid overfitting of the solutions to the training data, the validation sets were used as a comparator. Once candidate predictors had been developed, they were then manually applied to a verification data set to assess the predictor performance against data that has not been used at all to generate the predictor.

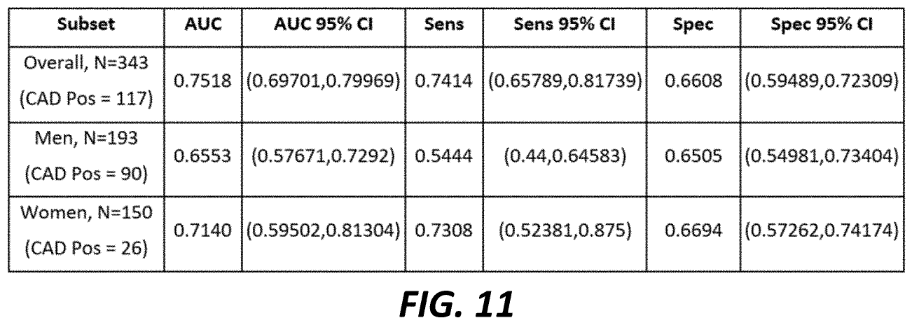

Results for predictors were computed on a test set of N=343 human subjects. FIG. 11 shows a table of diagnostic performance of the predictors using the exemplary system of FIG. 1. The figure shows overall result for sensitivity (“sens”) and specificity (“spec”) of the predictor as well as results as stratified by genders. An area under the curve (“AUC” in the table of FIG. 11) value greater than 0.5 corresponds to a higher a predictor performance. Similarly, the higher the specificity (“Spec” in the table of FIG. 11), the greater the ability to detect the negative (no-disease) cases, and the higher the sensitivity (“Sens” in the table of FIG. 11), the greater the ability to detect the positive (disease) cases. As shown in FIG. 11, with an AUC greater than 0.5, the performance of the predictor provides confidence that the exemplary analysis using volumetric object of a residue point-cloud model/data set has clinical value and utility. The table of FIG. 11 also includes 95% confidence intervals (“CI”) for a given sensitivity or specificity.

Modeling Results.

In another aspect of the study, experiments were conducted to quantify contributions of a resulting residue from physiological abnormalities, modeling error, or a combination of both. The study concluded that valuation information about the heart functionality is hidden in the residue. The basis, discussed herein and summarized again, is that an abnormality cannot be modeled (i.e., exhibiting predominantly deterministic chaos behavior and not quasi-periodic behavior), and thus would appear in the residue.

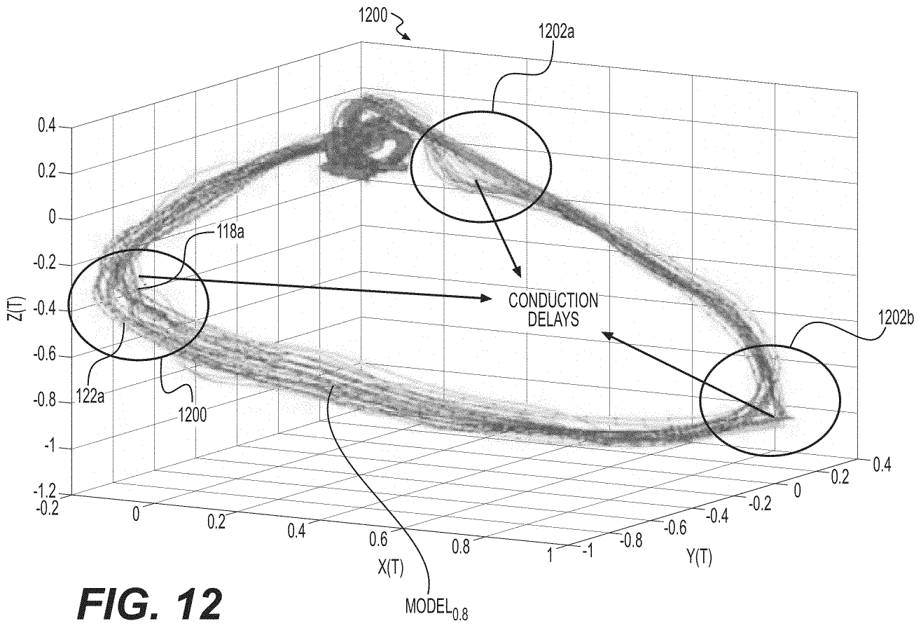

FIG. 12 shows a phase space plot 1200 that illustrates a set of subspace signals 118a (e.g., associated with the pre-processed biophysical signal data set) and corresponding subspace model 122a generated from the modeling module 120 (from which the module 124 can generate the residue point-cloud model/data set 125 (not shown)), in accordance with an illustrative embodiment. Indeed, plot 1200 shows (for example in regions denoted by reference circles 1202a, 1202b, and 1202c) differences between the subspace model 122a and the subspace signal 118a. These differences were hypothesized and validated in the CADLAD study to contain information about conduction delay.

In another aspect of the CADLAD study, the number of selected basis functions that collectively form the subspace model 122a were varied. In FIG. 12, 80% of the basis functions as would be optimally generated by the analysis module 120, using a sparse approximation algorithm, are co-presented with the subspace signal 118a in plot 1200. Indeed, by adjusting the number of basis functions that is included in the subspace model 122a, the difference can be modified.

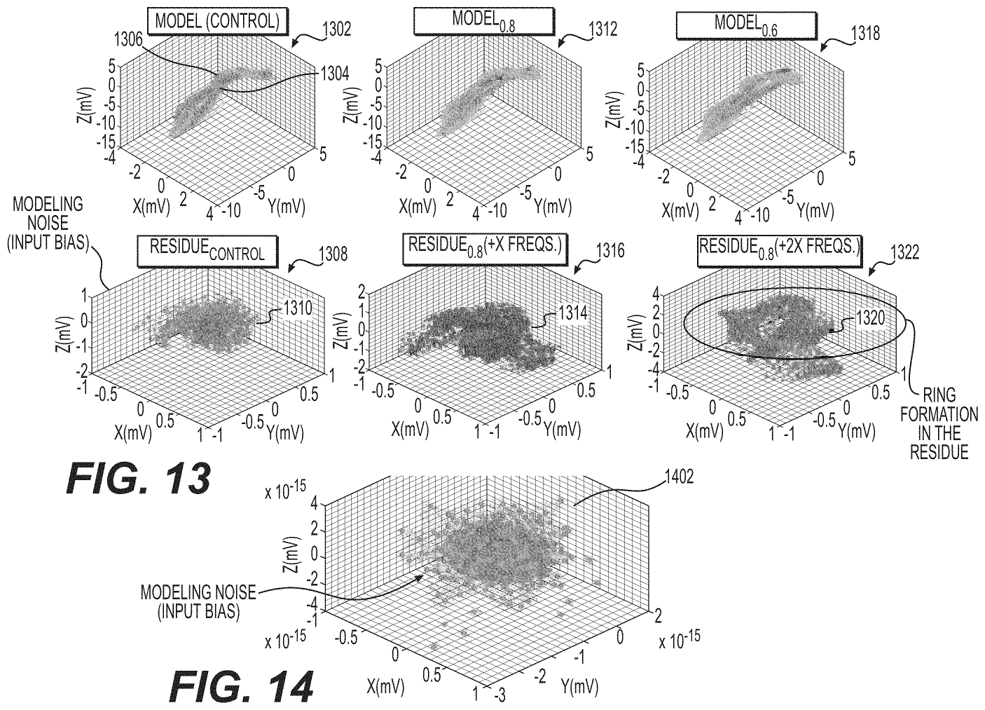

FIG. 13 shows differing residue pattern generated based on number of selected basis functions used to generate the residue subspace model 122a, in accordance with an illustrative embodiment. As shown in FIG. 13, a first plot 1302 shows an input subspace signal 1306 and a corresponding control subspace model 1304 generated from the subspace signal 1306 for a given experiment. FIG. 13 further shows a second plot 1308 of a resulting residue data set 125 (shown as 1310) generated from subtracting the subspace signal 1306 with the subspace model 1304. Two additional experiments are shown in which the number of basis functions that are included in the subspace model are varied. In plot 1312, corresponding to a second experiment, only 80% of the basis functions in the control subspace model 1304 were used to generate a residue data set (shown as 1314) in plot 1316. In plot 1318, corresponding to a third experiment, only 60% of the basis functions in the control subspace model 1304 were used to generate a residue data set (shown as 1320) in plot 1322. Indeed, in the second experiment, the residue data set 1314 include x number of additional frequencies components that are not included in the control residue 1310. Similarly, in the third experiment, the residue data set 1320 includes twice as many additional frequencies components as compared to the residue 1314 that are not included in the control residue 1310. In plot 1322, the residue data set 1320 appears to form a ring pattern, which is likely attributed to the quasi-periodic components in the input signal that are now included in the residue.

To further quantify this modeling noise, a study was conducted using a Fast Fourier Transform (“FFT”) technique or analysis to model the signal. It is generally understood that Fast Fourier Transform can be used to perfectly decompose and reconstruct a signal, but Fast Fourier Transform would model the noise as well. FIG. 14 shows a residue model/data set 1402 generated by modeling frequencies (e.g., 20,000 frequencies) in the input subspace signal 1306 using the Fast Fourier Transform. Because Fast Fourier Transform models everything, the residue data set 1402 represents the contribution from only the modeling noise. As shown in FIG. 14, the data has an order of 10E-15, which supports the conclusion that the residue is just modeling/numerical noise. A spectral density analysis was then performed to ascertain the modeling bias as a ratio of i) the difference between the power spectral energy of the sparse approximation algorithm and the power spectral density of the Fast Fourier Transform over the power spectral density of the Fast Fourier Transform per Equation 1:

$${PSD}_{model \_ bias \_ of \_ model} = \frac{{PSD}_{signal \_ FFT} - {PSD}_{model}}{{PSD}_{signal \_ FFT}} \qquad ( Equation 1 )$$

While a portion of the PSD fluctuations may be attributed to variations in noise level within the signal, such portion was removed in the residue analysis through bias removal. Per the CADLA study, therefore, it is concluded that a sparse approximation algorithm can induce modeling noise (in the residue) that are mostly inbounded with the residues in phase space. In conjunction with FFT analysis, the model noise can be quantified.

### Example Computing Device

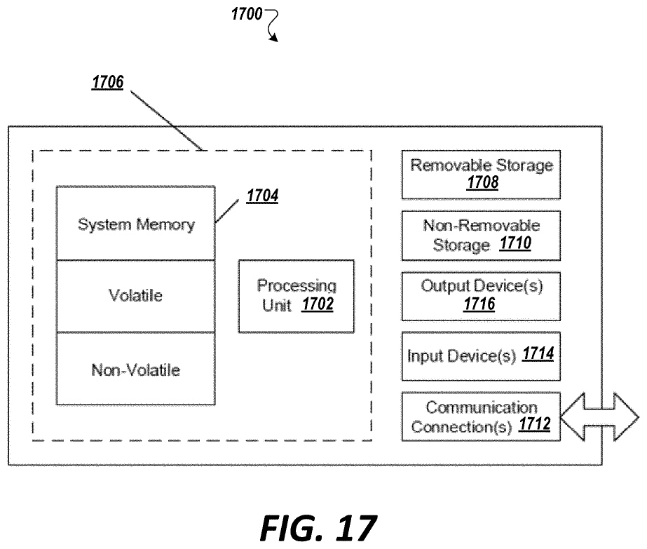

FIG. 17 shows an exemplary computing environment in which example embodiments of the assessment system 110 and aspects thereof may be implemented.

The computing device environment is only one example of a suitable computing environment and is not intended to suggest any limitation as to the scope of use or functionality.

Numerous other general-purpose or special purpose computing devices environments or configurations may be used. Examples of well-known computing devices, environments, and/or configurations that may be suitable for use include, but are not limited to, personal computers, server computers, handheld or laptop devices, mobile phones, wearable devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 17, an exemplary system for implementing aspects described herein includes a computing device, such as computing device 1700. In its most basic configuration, computing device 1700 typically includes at least one processing unit 1702 and memory 1704. Depending on the exact configuration and type of computing device, memory 1704 may be volatile (such as random-access memory (RAM)), non-volatile (such as read-only memory (ROM), flash memory, etc.), or some combination of the two. This most basic configuration is illustrated in FIG. 17 by dashed line 1706.

Computing device 1700 may have additional features/functionality. For example, computing device 1700 may include additional storage (removable and/or non-removable) including, but not limited to, magnetic or optical disks or tape. Such additional storage is illustrated in FIG. 17 by removable storage 1708 and non-removable storage 1710.

Computing device 1700 typically includes a variety of computer readable media. Computer readable media can be any available media that can be accessed by the device 1700 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 1704, removable storage 1708, and non-removable storage 1710 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 1700. Any such computer storage media may be part of computing device 1700.

Computing device 1700 may contain communication connection(s) 1712 that allow the device to communicate with other devices. Computing device 1700 may also have input device(s) 1714 such as a keyboard, mouse, pen, voice input device, touch input device, etc., singularly or in combination. Output device(s) 2716 such as a display, speakers, printer, vibratory mechanism, etc. may also be included singularly or in combination. All these devices are well known in the art and need not be discussed at length here.

It should be understood that the various techniques described herein may be implemented in connection with hardware components or software components or, where appropriate, with a combination of both. Illustrative types of hardware components that can be used include Graphical Processing Units (GPUs), Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, handheld devices, and wearable devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Further examples of various processing that may be used with the exemplified method and system are described in: U.S. Pat. No. 9,289,150, entitled “Non-invasive Method and System for Characterizing Cardiovascular Systems”; U.S. Pat. No. 9,655,536, entitled “Non-invasive Method and System for Characterizing Cardiovascular Systems”; U.S. Pat. No. 9,968,275, entitled “Non-invasive Method and System for Characterizing Cardiovascular Systems”; U.S. Pat. No. 8,923,958, entitled “System and Method for Evaluating an Electrophysiological Signal”; U.S. Pat. No. 9,408,543, entitled “Non-invasive Method and System for Characterizing Cardiovascular Systems and All-Cause Mortality and Sudden Cardiac Death Risk”; U.S. Pat. No. 9,955,883, entitled “Non-invasive Method and System for Characterizing Cardiovascular Systems and All-Cause Mortality and Sudden Cardiac Death Risk”; U.S. Pat. No. 9,737,229, entitled “Noninvasive Electrocardiographic Method for Estimating Mammalian Cardiac Chamber Size and Mechanical Function”; U.S. Pat. No. 10,039,468, entitled “Noninvasive Electrocardiographic Method for Estimating Mammalian Cardiac Chamber Size and Mechanical Function”; U.S. Pat. No. 9,597,021, entitled “Noninvasive Method for Estimating Glucose, Glycosylated Hemoglobin and Other Blood Constituents”; U.S. Pat. No. 9,968,265, entitled “Method and System for Characterizing Cardiovascular Systems From Single Channel Data”; U.S. Pat. No. 9,910,964, entitled “Methods and Systems Using Mathematical Analysis and Machine Learning to Diagnose Disease”; U.S. Publication No. 2017/0119272, entitled “Method and Apparatus for Wide-Band Phase Gradient Signal Acquisition”; PCT Publication No. WO2017/033164, entitled “Method and Apparatus for Wide-Band Phase Gradient Signal Acquisition”; U.S. Publication No. 2018/0000371, entitled “Non-invasive Method and System for Measuring Myocardial Ischemia, Stenosis Identification, Localization and Fractional Flow Reserve Estimation”; PCT Publication No. WO2017/221221, entitled “Non-invasive Method and System for Measuring Myocardial Ischemia, Stenosis Identification, Localization and Fractional Flow Reserve Estimation”; U.S. Pat. No. 10,292,596, entitled “Method and System for Visualization of Heart Tissue at Risk”; U.S. Publication No. 2018/0249960, entitled “Method and System for Wide-band Phase Gradient Signal Acquisition”; U.S. application Ser. No. 16/232,801, filed on Dec. 26, 2018, entitled “Method and System to Assess Disease Using Phase Space Volumetric Objects”; PCT Application No. IB/2018/060708, entitled “Method and System to Assess Disease Using Phase Space Volumetric Objects”; U.S. Patent Publication No. US2019/0117164, entitled “Methods and Systems of De-Noising Magnetic-Field Based Sensor Data of Electrophysiological Signals”; U.S. Publication No. 2019/0214137, filed on Dec. 26, 2018, entitled “Method and System to Assess Disease Using Phase Space Tomography and Machine Learning”; PCT Application No. PCT/IB32018/060709, entitled “Method and System to Assess Disease Using Phase Space Tomography and Machine Learning”; U.S. Publication No. 2019/0384757, entitled “Methods and Systems to Quantify and Remove Asynchronous Noise in Biophysical Signals,” filed Jun. 18, 2019; U.S. Publication No. 2019/0365265, entitled “Method and System to Assess Pulmonary Hypertension Using Phase Space Tomography and Machine Learning”; U.S. patent application Ser. No. ______, concurrently filed herewith, entitled “Method and System for Automated Quantification of Signal Quality” (having attorney docket no. 10321-036us1 and claiming priority to U.S. Patent Provisional Application No. 62/784,962); U.S. patent application Ser. No. ______, entitled “Method and System to Configure and Use Neural Network To Assess Medical Disease” (having attorney docket no. 10321-037pv1 and claiming priority to U.S. Patent Provisional Application No. 62/784,925); U.S. application Ser. No. 15/653,433, entitled “Discovering Novel Features to Use in Machine Learning Techniques, such as Machine Learning Techniques for Diagnosing Medical Conditions”; U.S. application Ser. No. 15/653,431, entitled “Discovering Genomes to Use in Machine Learning Techniques”; U.S. patent application Ser. No. ______ (claiming priority to application having attorney docket no. 10321-041pv1), entitled “Method and System to Assess Disease Using Dynamical Analysis of Cardiac and Photoplethysmographic Signals”); U.S. patent application Ser. No. ______ (claiming priority to application having attorney docket no. 10321-040pv1), entitled “Method and System to Assess Disease Using Dynamical Analysis of Biophysical Signals”, each of which is incorporated by reference herein in its entirety.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

While the methods and systems have been described in connection with certain embodiments and specific examples, it is not intended that the scope be limited to the particular embodiments set forth, as the embodiments herein are intended in all respects to be illustrative rather than restrictive.

The methods, systems and processes described herein may be used generate stenosis and FFR outputs for use in connection with procedures such as the placement of vascular stents within a vessel such as an artery of a living (e.g., human) subject, and other interventional and surgical system or processes. In one embodiment, the methods, systems and processes described herein can be configured to use the FFR/stenosis outputs to determine and/or modify, intra operation, a number of stents to be placed in a living (e.g., human), including their optimal location of deployment within a given vessel, among others.

Examples of other biophysical signals that may be analyzed in whole, or in part, using the exemplary methods and systems include, but are not limited to, an electrocardiogram (ECG) data set, an electroencephalogram (EEG) data set, a gamma synchrony signal data set; a respiratory function signal data set; a pulse oximetry signal data set; a perfusion data signal data set; a quasi-periodic biological signal data set; a fetal ECG data set; a blood pressure signal; a cardiac magnetic field data set, and a heart rate signal data set.

The exemplary analysis can be used in the diagnosis and treatment of cardiac-related pathologies and conditions and/or neurological-related pathologies and conditions, such assessment can be applied to the diagnosis and treatment (including, surgical, minimally invasive, and/or pharmacologic treatment) of any pathologies or conditions in which a biophysical signal is involved in any relevant system of a living body. One example in the cardiac context is the diagnosis of CAD and its treatment by any number of therapies, alone or in combination, such as the placement of a stent in a coronary artery, performance of an atherectomy, angioplasty, prescription of drug therapy, and/or the prescription of exercise, nutritional and other lifestyle changes, etc. Other cardiac-related pathologies or conditions that may be diagnosed include, e.g., arrhythmia, congestive heart failure, valve failure, pulmonary hypertension (e.g., pulmonary arterial hypertension, pulmonary hypertension due to left heart disease, pulmonary hypertension due to lung disease, pulmonary hypertension due to chronic blood clots, and pulmonary hypertension due to other disease such as blood or other disorders), as well as other cardiac-related pathologies, conditions and/or diseases. Non-limiting examples of neurological-related diseases, pathologies or conditions that may be diagnosed include, e.g., epilepsy, schizophrenia, Parkinson's Disease, Alzheimer's Disease (and all other forms of dementia), autism spectrum (including Asperger syndrome), attention deficit hyperactivity disorder, Huntington's Disease, muscular dystrophy, depression, bipolar disorder, brain/spinal cord tumors (malignant and benign), movement disorders, cognitive impairment, speech impairment, various psychoses, brain/spinal cord/nerve injury, chronic traumatic encephalopathy, cluster headaches, migraine headaches, neuropathy (in its various forms, including peripheral neuropathy), phantom limb/pain, chronic fatigue syndrome, acute and/or chronic pain (including back pain, failed back surgery syndrome, etc.), dyskinesia, anxiety disorders, conditions caused by infections or foreign agents (e.g., Lyme disease, encephalitis, rabies), narcolepsy and other sleep disorders, post-traumatic stress disorder, neurological conditions/effects related to stroke, aneurysms, hemorrhagic injury, etc., tinnitus and other hearing-related diseases/conditions and vision-related diseases/conditions.

What is claimed is:

1. A method for non-invasively assessing a representation of a physiological system in which the representation is indicative of a disease state of a subject, the method comprising:
obtaining, by one or more processors, a plurality of biophysical signal data sets of a subject;
generating, by the one or more processors, a residue model from an analysis of the plurality of biophysical signal data sets;
generating, by the one or more processors, a three-dimensional volumetric object from a point-cloud residue, wherein the point-cloud residue comprises a plurality of vertices defined in a three-dimensional phase space of the plurality of biophysical signal data sets; and
determining, by the one or more processors, machine extractable features associated with a geometric associated aspect of the three-dimensional volumetric object, wherein the one or more machine extractable features are used as an indicator of a disease state.

2. The method of claim 1, wherein the step of generating the three-dimensional volumetric object comprises:
performing a triangulation operation on the point-cloud residue of the plurality of biophysical signal data sets, wherein the triangulation operation is selected from the group consisting of Delaunay triangulation, Mesh generation, Alpha Hull triangulation, and Convex Hull triangulation.

3. The method of claim 1, wherein the machine extractable features are used in a machine-trained estimation of presence and/or non-presence of significant coronary artery disease.

4. The method of claim 1, wherein the machine extractable features are selected from the group consisting of a 3D object volume value, a void volume value, a surface area value, a principal curvature direction value, and a Betti number value.

5. The method of claim 1, further comprising:
generating a contour data set for each tomographic image of the set of tomographic images, wherein the contour data are presented for the assessment of presence and/or non-presence of significant coronary artery disease.

6. The method of claim 1, wherein the acquired plurality of biophysical signal data sets are derived from measurements acquired via a noninvasive equipment configured to measure properties selected from the group consisting of electric properties, magnetic properties, acoustic properties, impedance properties, and reflectance properties of a physiological system.

7. The method of claim 1 further comprising:
removing, by the one or more processors, a baseline wandering trend from the acquired data prior to generating the plurality of models.

8. The method of claim 1 comprising:
causing, by the one or more processors, generation of a visualization of generated volumetric object as a three-dimensional object, wherein the three-dimensional object is rendered and displayed at a display of a computing device and/or presented in a report.

9. The method of claim 1, wherein each of the acquired biophysical signal data sets comprises a wide-band phase gradient biopotential signal data set that is simultaneously acquired at a sampling rate selected from the group consisting of about 1 kHz, about 2 kHz, about 3 kHz, about 4 kHz, about 5 kHz, about 6 kHz, about 7 kHz, about 8 kHz, about 9 kHz, about 10 kHz, and greater than 10 kHz.

10. The method of claim 1, wherein the residue model is generated by:
a subtraction operation of the acquired biophysical signal data sets and a data set generated from the analysis of the plurality of biophysical signal data sets.

11. The method of claim 1, wherein the analysis of the plurality of biophysical signal data sets comprises a quasi-periodic analysis of the frequency components of the plurality of biophysical signal data sets.

12. The method of claim 1, wherein the analysis of the plurality of biophysical signal data sets comprises a chaotic analysis of the frequency components of the plurality of biophysical signal data sets.

13. The method of claim 1, wherein the analysis of the plurality of biophysical signal data sets comprises a phase analysis of the plurality of biophysical signal data sets.

14. A system comprising:
a processor; and
a memory having instructions thereon, wherein the instructions when executed by the processor, cause the processor to:
obtain a plurality of biophysical signal data sets of a subject;
generate a residue model from an analysis of the plurality of biophysical signal data sets;
generate a three-dimensional volumetric object from a point-cloud residue, wherein the point-cloud residue comprises a plurality of vertices defined in a three-dimensional phase space of the plurality of biophysical signal data sets; and
determine machine extractable features associated with a geometric associated aspect of the three-dimensional volumetric object, wherein the one or more machine extractable features are used as an indicator of a disease state.

15. The system of claim 14, wherein the instruction to generate the three-dimensional volumetric object comprises:
instructions to perform a triangulation operation on the point-cloud residue of the plurality of biophysical signal data sets, wherein the triangulation operation is selected from the group consisting of Delaunay triangulation, Mesh generation, Alpha Hull triangulation, and Convex Hull triangulation.

16. The system of claim 14, wherein the machine extractable features are selected from the group consisting of a 3D object volume value, a void volume value, a surface area value, a principal curvature direction value, and a Betti number value.

17. The system of claim 14 further comprising:
a noninvasive equipment configured to measure properties selected from the group consisting of electric properties, magnetic properties, acoustic properties, impedance properties, and reflectance properties of a physiological system.

18. The system of claim 14, wherein the noninvasive equipment comprises a phase space recorder and/or an optical photoplethysmograph system.

19. The system of claim 14, wherein the analysis of the plurality of biophysical signal data sets comprises at least one of: a quasi-periodic analysis of the frequency components of the plurality of biophysical signal data sets, a chaotic analysis of the frequency components of the plurality of biophysical signal data sets, and a phase analysis of the plurality of biophysical signal data sets.

20. A non-transitory computer readable medium having instructions stored thereon, wherein execution of the instructions, cause the processor to:
obtain a plurality of biophysical signal data sets of a subject;
generate a residue model from an analysis of the plurality of biophysical signal data sets;
generate a three-dimensional volumetric object from a point-cloud residue, wherein the point-cloud residue comprises a plurality of vertices defined in a three-dimensional phase space of the plurality of biophysical signal data sets; and
determine machine extractable features associated with a geometric associated aspect of the three-dimensional volumetric object, wherein the one or more machine extractable features are used as an indicator of a disease state.
